(19) United States
(12) Patent Application Publication
(10) Pub. No.: US 2026/0144566 A1
(43) Pub. Date: May 28, 2026

(54) CERVICAL DISC INSTRUMENTS

(71) Applicant: GLOBUS MEDICAL, INC., AUDUBON, PA (US)

(72) Inventors: Michael Brotman, San Diego, CA (US); Andrew Hartman, San Diego, CA (US); Byron Riemhofer, San Diego, CA (US); Jackson Feld, San Diego, CA (US); Elijah Lettau, Lakeside, CA (US); Rob German, San Diego, CA (US); Prashant Tambey, San Diego, CA (US)

(21) Appl. No.: 19/453,707

(22) Filed: Jan. 20, 2026

Related U.S. Application Data
(62) Division of application No. 18/788,499, filed on Jul. 30, 2024.
Application No. 18/788,499 is a continuation of application No. 18/788,437, filed on Jul. 30, 2024.

(51) Int. Cl. A61B 17/3205; A61B 17/00; A61B 17/32; A61F 2/44; A61F 2/46

(52) U.S. Cl. CPC A61B 17/3205; A61B 17/320016; A61F 2/4425; A61F 2/4684; A61B 2017/00261; A61B 2017/320052

(57) ABSTRACT

A cervical disc instrument that includes a handle that defines a cavity therein, a trial head, and a blade. The trial head is removably coupled to the handle and configured for insertion into a disc space. The blade is disposed in the cavity of the handle and configured to cut a surface that at least partially defines the disc space when the trial head is located in the disc space.

### CROSS-REFERENCE TO RELATED APPLICATIONS

The present application is a divisional application of U.S. patent application Ser. No. 18/788,499 filed on Jul. 30, 2024, which is a continuation of U.S. patent application Ser. No. 18/788,437, filed Jul. 30, 2024, the entire contents of each of which is incorporated herein by reference in its entirety for all purposes.

### TECHNICAL FIELD

This disclosure relates to cervical disc instruments, and more particularly, cervical disc instruments for disc replacement.

### BACKGROUND

Total disc replacement (TDR) surgery may be completed for a patient suffering from various spinal conditions, such as degenerative disc disease, whereby the patient may have degeneration of intervertebral discs in their spine. Such degeneration may cause chronic pain, reduced mobility, diminished quality of life, or a combination thereof for the patient. TDR may provide an alternative procedure for more traditional treatment methods, such as spinal fusion, in which the patient may exhibit limitations to spinal motion and potential complications in adjacent spinal segments.

To prepare for TDR surgery, a slot may be cut into the superior and inferior endplates (e.g., the upper surface of the vertebral body located above the intervertebral disc being replaced and the lower surface of the vertebral body located below the intervertebral disc being replaced). The keels of the disc implant may align with and follow the aforementioned slots. Thus, the slots cut into the superior and inferior endplates may determine a final trajectory of the disc implant.

Conventionally, more than one instrument may be required to complete the TDR surgery. By way of example, a first instrument may be required to determine a proper location for the disc implant and a second instrument may be required to cut the slots in the superior and inferior endplates based upon the prior location determined by the first instrument. It is often critical to ensure that the determined location (e.g., the proper location for the disc implant) is located properly within the disc space along a midline of the disc space with respect to a lateral direction of the disc space. Similarly, it is often critical to ensure that the slots cut in the superior and inferior endplates align with the trialed location and are cut to a proper depth to avoid damage to nerve roots or the spinal cord of the patient.

### SUMMARY

In one implementation, a cervical disc instrument includes a handle that defines a cavity therein, a trial head, and a blade. The trial head is removably coupled to the handle and configured for insertion into a disc space. Additionally, the blade is disposed in the cavity of the handle and configured to cut a surface that at least partially defines the disc space when the trial head is located in the disc space.

In another implementation, a cervical disc instrument includes a handle, a trial head, and a blade. The trial head includes a trial head shaft. The trial head is configured for insertion into a disc space. Additionally, the trial head is removably coupled to the handle by the trial head shaft. Moreover, the blade is disposed in a groove of the trial head shaft and configured to cut a surface that at least partially defines the disc space when the trial head is located in the disc space.

In another implementation, a cervical disc instrument includes a handle, a trial head removably coupled to the handle by a trial head shaft extending therebetween, and a blade disposed in a groove of the trial head shaft. The blade is configured to move along the groove of the trial head shaft. Additionally, the blade is configured for insertion into the groove through a keyed opening and is retained in the groove by a retention feature.

In another implementation, a cervical disc instrument includes a handle, a trial head removably coupled to the handle and a blade component movably coupled to the handle. The trial head is configured for insertion into a disc space. Additionally, a groove of the handle is configured to guide the blade along the groove of the handle.

In another implementation, a cervical disc instrument includes a handle that includes a groove along an outer surface of the handle, a trial head removably coupled to the handle, a blade component slidably coupled to the handle, and a depth stop. The trial head is configured for insertion into a disc space. Additionally, the groove is configured to guide the blade component along the groove. Moreover, the depth stop is configured to define a cutting depth of the blade with respect to a surface that at least partially defines the disc space, whereby the depth stop, the handle, the trial head, and the blade component are coaxial along a longitudinal axis of the cervical disc instrument.

In another implementation, the cervical disc instrument includes a handle that include a groove along an outer surface of the handle, a trial head removably coupled to the handle by a latch mechanism, and a blade component movably coupled to the handle. The blade component is configured to guide along the groove. Additionally, the blade component at least partially encloses the handle.

In another implementation, a cervical disc instrument is configured to insert a disc implant into a disc space. The cervical disc instrument includes a jaw component, a sheath, a drive shaft, and a tamp. The jaw component includes a jaw shaft defining a first cavity therein and jaws extending from the jaw shaft, whereby the jaws are configured to grasp the disc implant. The sheath defines a second cavity therein and the jaw shaft is positioned in the second cavity. Additionally, the drive shaft is configured to extend through the first cavity and the second cavity and the tamp is coupled to the drive shaft and positioned between the jaws.

In another implementation, a cervical disc replacement system includes a disc implant and a cervical disc instrument that is configured to insert the disc implant into a disc space. The cervical disc instrument includes a jaw component, a sheath at least partially surrounding the jaw component, a drive shaft configured to extend through the sheath, and a tamp coupled to the drive shaft. The jaw component is configured to grasp the disc implant for insertion of the disc implant into the disc space. The sheath is configured to move the jaw component to grasp the disc implant. Additionally, the tamp is configured to translate along a longitudinal axis of the cervical disc instrument to release the disc implant from the jaw component.

In another implementation, a cervical disc instrument includes a jaw component, a sheath configured to at least partially surround the jaw component, a drive shaft configured to extend through the sheath, a tamp coupled to the drive shaft, and a depth stop. The jaw component is configured to releasably engage a disc implant for insertion of the disc implant into a disc space. The tamp is configured to translate along a longitudinal axis of the cervical disc instrument to release the disc implant from the jaw component. Additionally, the depth stop is configured to prevent over-insertion of the disc implant into the disc space.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1A is a perspective view of a first example of a cervical disc instrument.

FIG. 1B is an exploded view of the cervical disc instrument shown in FIG. 1A.

FIG. 2 is a close-up perspective view of the cervical disc instrument shown in FIG. 1A.

FIG. 3 is a close-up perspective view of a blade of the cervical disc instrument shown in FIG. 1A.

FIG. 4 is a perspective view of an example of a trial head shaft of a cervical disc instrument.

FIG. 5 is a cross-sectional view of the cervical disc instrument shown in FIG. 1A.

FIG. 6 is an exploded view of a second example of a cervical disc instrument.

FIG. 7 is a partial cross-sectional view of the cervical disc instrument shown in FIG. 6.

FIG. 8 is a close-up cross-sectional view of the latch mechanism shown in FIG. 7.

FIG. 9 is a cross-sectional view of the cervical disc instrument shown in FIG. 6.

FIG. 10A is an exploded view of a third example of a cervical disc instrument.

FIG. 10B is another exploded view of the cervical disc instrument shown in FIG. 10A.

FIG. 11 is a close-up perspective view of the cervical disc instrument shown in FIG. 10A.

FIG. 12 is a cross-sectional view of the cervical disc instrument shown in FIG. 10A.

FIG. 13A is a perspective view the cervical disc instrument shown in FIG. 10A prior to insertion of the blade into the cervical disc instrument.

FIG. 13B is a perspective view of the cervical disc instrument shown in FIG. 13A after insertion of the blade into the cervical disc instrument.

FIG. 14 is an exploded view of a fourth example of a cervical disc instrument.

FIG. 15 is another exploded view of the cervical disc instrument shown in FIG. 15.

FIG. 16 is a close-up perspective view of a handle of the cervical disc instrument shown in FIG. 14.

FIG. 17 is an exploded view of the handle shown in FIG. 16.

FIG. 18A is a perspective view of a fifth example of a cervical disc instrument.

FIG. 18B is another perspective view of the cervical disc instrument shown in FIG. 18A prior to the cervical disc instrument coupling to a disc implant.

FIG. 19 is a perspective view of the cervical disc instrument shown in FIGS. 18A and 18B coupled to the disc implant shown in FIG. 18B.

FIG. 20 is an exploded view of the cervical disc instrument shown in FIG. 18A.

FIG. 21 is a cross-sectional view of the cervical disc instrument shown in FIG. 18A.

FIG. 22 is a close-up cross-sectional view of the cervical disc instrument shown in FIG. 21.

FIG. 23A is a perspective view of a sixth example of a cervical disc instrument having a clip coupled to a disc implant and disconnected from the cervical disc instrument.

FIG. 23B is a perspective view of the cervical disc instrument shown in FIG. 23A having the clip coupled to the disc implant and coupled to the cervical disc instrument.

FIG. 23C is a perspective view of the cervical disc instrument shown in FIGS. 23A and 23B having the clip coupled to the cervical disc instrument and disconnected from the disc implant.

FIG. 24 is a close-up perspective view of the clip coupled to the disc implant shown in FIG. 23A.

FIG. 25A is a top-down view of an example of a disc implant.

FIG. 25B is a perspective view of the disc implant shown in FIG. 25A.

FIG. 26 is a perspective view of the disc implant shown in FIG. 25A coupled to the clip of the cervical disc instrument shown in FIG. 23A.

### DETAILED DESCRIPTION

Total disc replacement (TDR) surgery may be completed on a patient suffering from damaged and/or degenerated cervical discs. To complete the TDR surgery, a surgeon may remove the damaged and/or deteriorated cervical disc and replace it with an artificial implant (e.g., a disc implant), thereby restoring normal function and motion to the affected area of the spine while also reducing pain. When the damaged and/or deteriorated cervical disc is removed, the disc space (e.g., a space from where the damaged and/or deteriorated disc is removed) may require preparation in order to properly receive the disc implant. For example, one or more slots may be cut into bone that at least partially surrounds the disc space (e.g., slots cut into a superior and/or inferior endplate positioned adjacent to the disc space) such that a portion of the disc implant (e.g., keels of the disc implant) may follow the prepared slots to reach its final position within the disc space. Additionally, trialing of a test implant may also be required to determine a proper location, size, fit, or a combination thereof of the final disc implant and/or to determine a proper location of the slots cut into the bone.

To facilitate preparation of the disc space and trialing of the test implant, current TDR surgery procedures and workflows may require that the surgeon complete trialing (e.g., trialing of the test implant) and cutting separately. That is, the surgeon may be required to use a first instrument to trial the proper location for the disc implant and a separate instrument to cut the slots to guide and/or position the disc implant. As such, the surgeon may be unable to cut the slots into the bone while the test implant (e.g., a trial head) is positioned within the disc space, even though a position of the slots may be determined by the location of the trial head. Additionally, conventional instruments used for trialing may often obstruct a view of the disc space, thereby hindering the surgeon's ability to properly locate the trial implant within the disc space. For example, anteroposterior fluoroscopy may be used during the TDR surgery to capture images of the disc space when the trial implant is located in the disc space. However, due to the size and/or shape of the conventional instruments used for trialing, a portion of such instruments may obstruct a view of the fluoroscopy imaging device.

The cervical disc instruments described herein improve on the procedure and workflow of the aforementioned TDR surgery. The cervical disc instruments may provide modularity between the test implant (e.g., the trial head) and a handle to ensure that imaging of the disc space remains unobstructed. Additionally, the cervical disc instruments may eliminate one or more steps within the procedure and workflow to further optimize the TDR surgery. For example, the cervical disc instruments described herein may integrate one or more conventionally separate instruments into a single instrument. It should also be noted that, while TDR surgery is described in further detail herein, the cervical disc instruments may also be configured for use in other types of operations, such as disc replacement of a disc not located within the cervical spine (e.g., thoracic discs and/or lumbar discs).

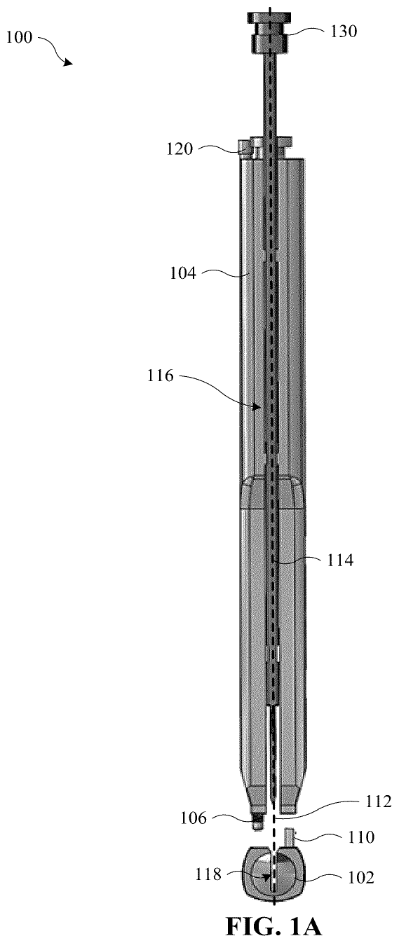

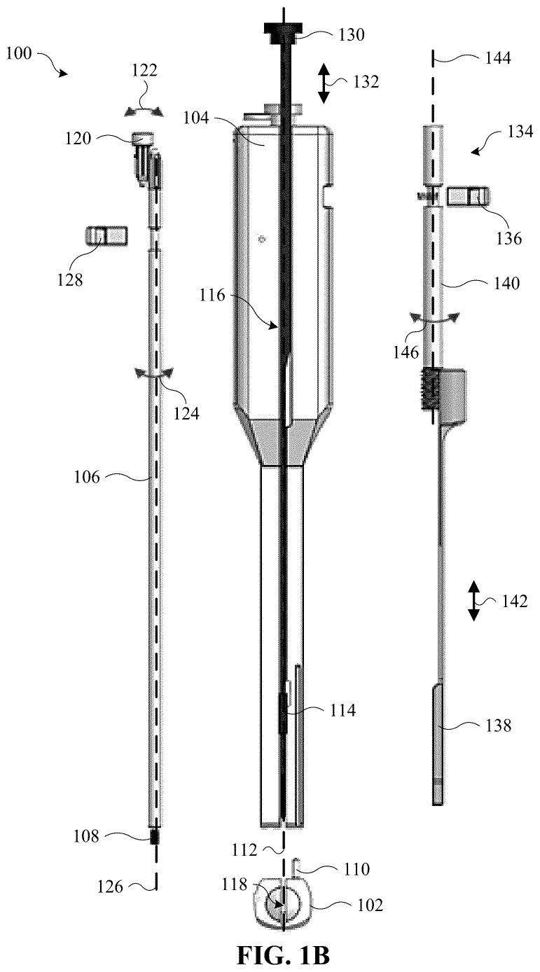

Turning now to the figures, FIG. 1A is a perspective view of a first example of a cervical disc instrument 100. FIG. 1B is an exploded view of the cervical disc instrument 100 shown in FIG. 1A. The cervical disc instrument 100 may be configured for trialing of a trial head 102 and/or may be configured for cutting of a surface that at least partially defines a disc space when the trial head 102 is located in the disc space. As described above, such disc space may be a location from where a damaged and/or deteriorated cervical disc is removed for replacement with a disc implant.

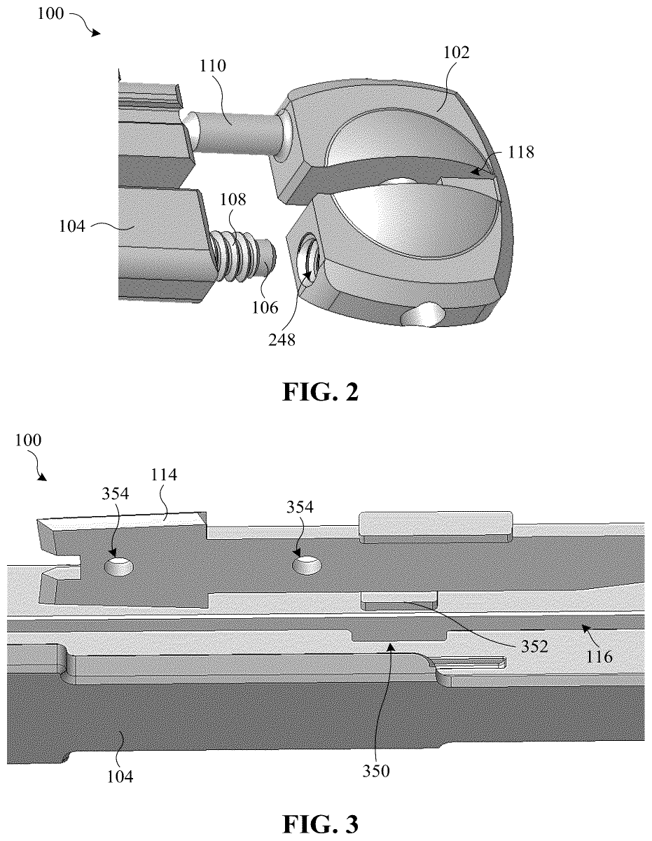

The cervical disc instrument 100 may include a handle 104 that is removably coupled to the trial head 102. For example, the trial head 102 may be coupled to the handle 104 by a trial head shaft 106 extending therebetween. As shown in further detail in FIG. 3, the trial head shaft 106 may include threading 108 that is received by a mounting hole of the trial head 102 to couple the trial head 102 to the handle 104. Additionally, to ensure proper alignment between the trial head 102 and the handle 104, the trial head 102 may include an alignment post 110 that may extend into an alignment hole of the handle 104. As such, the alignment post 110 may also ensure proper orientation of the trial head 102 and the handle 104 with respect to one another along a longitudinal axis 112 of the cervical disc instrument 100. Moreover, the trial head shaft 106 and the alignment post 110 may maintain a position of the trial head 102 such that the trial head 102 and the handle 104 are coaxial along the longitudinal axis 112 of the cervical disc instrument 100.

The cervical disc instrument 100 may also include a blade 114. The blade 114 may be configured to cut a surface (e.g., a surface of bone, such as superior and/or inferior endplates at the operative level with respect to the disc space) that at least partially defines the disc space. The blade 114 may be at least partially disposed in a cavity 116 of the handle 104 and configured to cut the surface when the trial head 102 is located in the disc space. For example, the blade 114 may extend along the longitudinal axis 112 of the cervical disc instrument 100 and move along the longitudinal axis 112 of the cervical disc instrument 100 to extend beyond a terminal end of the trial head 102, thereby allowing the blade 114 to contact the surface. The trial head 102 may also include a groove 118, and the blade 114 may extend through the groove 118 to contact the surface. As a result, cervical disc instrument 100 may facilitate trialing of the trial head 102 within the disc space while also allowing for cutting of the surface (e.g., via the blade 114) when the trial head 102 is located in the disc space.

To further detail operation of the cervical disc instrument 100, FIGS. 1A and 1B will now be described. To connect the trial head 102 to the handle 104, the alignment post 110 of the trial head 102 may be aligned with an alignment hole of the handle 104. Before, during, or after alignment of the alignment post 110, the trial head shaft 106 may be driven so that the threading 108 of the trial head shaft 106 may engage a mounting hole of the trial head 102 (e.g., threading within the mounting hole 248 of the trial head 102 as shown in FIG. 2.), thereby coupling the trial head 102 to the handle 104.

To drive the trial head shaft 106, a user (e.g., a surgeon) may drive (e.g., rotate) a drive pinion 120 in a direction 122. The drive pinion 120 may include teeth or other meshing that engages respective teeth or meshing of the trial head shaft 106 such that rotation of the drive pinion 120 in the direction 122 may drive rotation of the trial head shaft 106 in a direction 124 about an axis of rotation 126 of the trial head shaft 106. As a result, the threading 108 may rotate to engage the trial head 102 and connect the trial head 102 to the handle 104. It should also be noted that the trial head shaft 106 and/or the drive pinion 120 may be removably connected to the handle 104 by a fastener 128. The fastener 128 may be a clip, latch, pin, bolt, other mechanical engaging feature, or a combination thereof that secures the trial head shaft 106 and/or the drive pinion 120 to the handle 104.

Once the trial head 102 is coupled to the handle 104, the trial head 102 may be inserted into the disc space to determine a proper position (e.g., location and/or orientation) of the trial head 102 within the disc space. After positioning of the trial head 102, the blade 114 may be used to cut a surface of the bone surrounding the disc space. The blade 114 is not particularly limited to any type of cutting and may be used to perform any operation needed within the disc space.

By way of example, the blade 114 may include, or may be coupled to, an impact cap 130 that extends outward (e.g., positioned outside of) the handle 104. The impact cap 130 may be pressed (e.g., impacted) to move the blade 114 in a direction 132 towards the trial head 102 and along the groove 118 of the trial head 102. As a result, the blade 114 may cut one or more slots along the surface—or surfaces—that at least partially extend around the disc space, whereby a disc implant may be guided along the slots. Thus, the blade 114 may cut the slots in a precise location based upon the positioning of the trial head 102 within the disc space without removing the trial head 102 from its proper location.

To ensure that the slots or other cuts are completed at a desired depth, the cervical disc instrument 100 may also include a depth stop 134. The depth stop 134 may be removably coupled to the handle 104 via a fastener 136 of the depth stop 134, which may be similar or different than the fastener 128 of the trial head shaft 106. The depth stop 134 may be configured to define a cutting depth of the blade 114 with respect to the surface (e.g., the bone surface). The depth stop 134 may also be configured to prevent over-travel of the blade 114 (e.g., in the direction 132 towards the trial head 102), thereby preventing the blade 114 from cutting too deep and causing injury or permanent disability to the patient.

To facilitate such protection, the depth stop 134 may include a depth stop post 138 that is configured to contact the surface (e.g., the bone surface) or another surface in proximity to the surface being cut. A drive shaft 140 may be coupled to, or in communication with, the depth stop post 138 to drive the depth stop post 138 in a direction 142 towards and away from the trial head 102. The depth stop post 138 may translate in the direction 142 beyond the trial head 102 to ensure contact with the surface. Additionally, movement of the depth stop post 138 in the direction 142 may dictate a cutting depth of the blade 114. That is, the depth stop post 138 may be driven in the direction 142 by the drive shaft 140 to a desired position, whereby the drive shaft 140 may maintain the desired position of the depth stop post 138 while cutting the surface with the blade 114. As such, a position of the depth stop post 138—and thus a cutting depth of the blade 114—may be adjusted based upon a given application.

To drive the depth stop post 138, the drive shaft 140 may be rotated about an axis of rotation 144 of the drive shaft 140 in a direction 146. The drive shaft 140 may be in communication with the depth stop post 138, such as via teeth or other types of meshing, such that rotation of the drive shaft 140 about the axis of rotation 144 of the drive shaft 140 may translate the depth stop post 138 in the direction 142 towards and away from the drive shaft 140. Thus, adjustment of the depth stop 134 may be done either manually or via a secondary tool during operation of the cervical disc instrument 100.

FIG. 2 illustrates a close-up perspective view of the cervical disc instrument 100 shown in FIGS. 1A and 1B. In particular, FIG. 2 illustrates a close-up view of the trial head 102 prior to coupling the trial head 102 to the handle 104 of the cervical disc instrument 100.

As shown in FIG. 2, and as described above, the trial head 102 may be coupled to the handle 104 via a trial head shaft 106. The trial head shaft 106 may be coupled to the handle 104 and include the threading 108, whereby the threading 108 may be received by a mounting hole 248 of the trial head 102 to engage internal threading of the mounting hole 248. As a result, the trial head 102 may be coupled to the trial head shaft 106 to thereby couple the trial head 102 to the handle 104. It should be noted that any coupling means may be utilized to couple the trial head 102 to the handle 104. For example, mechanical fastening means other than threading may maintain engagement between the trial head 102 and the handle 104. Alternatively, or additionally, the trial head 102 may be magnetically coupled to the trial head shaft 106.

Additionally, as described above, the trial head 102 may include the alignment post 110. The alignment post 110 may project from the trial head 102 and extending into an alignment hole or other receiving feature of the handle 104. The alignment post 110 may be positioned anywhere along the trial head 102, such as on an opposing side of the groove 118 with respect to the mounting hole 248. As such, the alignment post 110 may maintain proper orientation of the trial head 102 with respect to the handle 104, such as orientation about the longitudinal axis 112 of the cervical disc instrument 100.

FIG. 3 illustrates a close-up perspective view of the blade 114 of the cervical disc instrument 100. As discussed above, the blade 114 may be inserted into (e.g., received by) the cavity 116 of the handle 104 so that the blade 114 may advance (e.g., move) along the longitudinal axis 112 of the cervical disc instrument 100.

The blade 114 may be inserted into the cavity 116 of the handle 104 through a keyed opening 350 defined by the cavity 116. The keyed opening 350 may be keyed (e.g., shaped) in a complementary manner to a keying feature 352 of the blade 114. As a result, the keyed opening 350 and the keying feature 352 may ensure that the blade 114 is inserted into the cavity 116 of the handle 104 in a proper location and with a proper orientation. Moreover, the keying feature 352 of the blade 114 may retain the blade 114 within the cavity 116. For example, the cavity 116 may be defined at least partially by one or more flanges and the keying feature 352 of the blade 114 may engage the one or more flanges to prevent the blade 114 from being removed from the cavity 116.

The blade 114 may also include one or more apertures 354. The apertures 354 may be positioned anywhere along the blade 114. For example, the apertures 354 may be disposed along the blade 114 such that the apertures 354 are positioned within the cavity 116 of the handle 104 when the blade 114 is inserted into the cavity 116. The apertures 354 may be positioned and configured to receive or otherwise engage a retention feature of the handle 104 that may be configured to retain a position of the blade 114 along the longitudinal axis 112 of the cervical disc instrument 100. For example, the retention feature may be a pin, spring, plunger, or other type of mechanical feature that may be received at least one of the apertures 354 to maintain a position of the blade 114 along the longitudinal axis 112 of the cervical disc instrument 100. The retention feature may then be released in any desired manner to allow for movement of the blade 114 in the direction 132 along the longitudinal axis 112 of the cervical disc instrument 100.

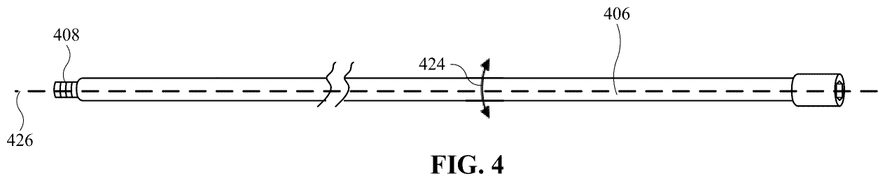

FIG. 4 illustrates a perspective view of another example of a trial head shaft 406, which may be similar to the trial head shaft 106 described above unless otherwise stated. The trial head shaft 406 may be implemented in the cervical disc instrument 100 described above in as an alternative to the trial head shaft 106.

While the trial head shaft 106 may be driven using the drive pinion 120, the trial head shaft 406 may not require a drive pinion 120 similar to the drive pinion 120. That is, while the trial head shaft 106 is indirectly rotated about the axis of rotation 126 of the trial head shaft 106 via rotating the drive pinion 120, the trial head shaft 406 may be directly driven (e.g., rotated) in a direction 424 about an axis or rotation 426 of the trial head shaft 406. As such, the drive pinion 120 may no longer be required, thereby improving the packaging space and potentially the operation of the cervical disc instrument 100.

It should also be noted that the trial head shaft 406 may be coupled to the trial head 102 in a similar manner to the trial head shaft 106. For example, the trial head shaft 406 may also include threading 408 similar to the threading 108, whereby the threading 408 may be received by and engage the mounting hole 248 of the trial head 102.

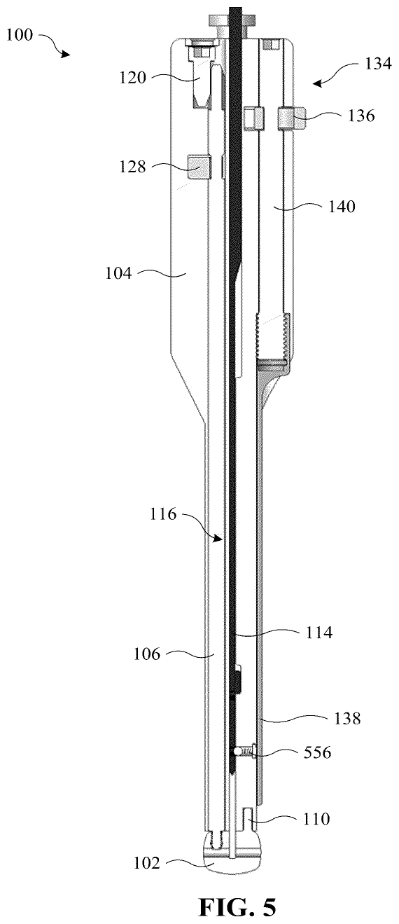

FIG. 5 illustrates a cross-sectional view of the cervical disc instrument 100 to further illustrate the inner componentry of the cervical disc instrument 100. As described above, the cervical disc instrument 100 may include the trial head 102 removably coupled to the handle 104 via the trial head shaft 106. The blade 114 may be disposed in the cavity 116 of the handle 104 and configured to move towards and away from the trial head 102 to cut a surface (e.g., a bone surface), whereby the depth stop 134 may be configured to prevent over-travel of the blade 114 to prevent the blade 114 from cutting too deep.

The blade 114 may be manually or electromechanically moved within the cavity 116. For example, during manual operation of the blade 114, the blade 114 may start in a first position, such as the position shown in FIG. 5. The first position may be considered a retracted position in which the blade 114 is not exposed outside of the handle 104. However, any position may be considered a first position. The blade 114 may retained in the first position via a retention feature 556 that extends into one or more of the apertures 354 of the blade 114. As shown in FIG. 5, the retention feature 556 may be a pin or plunger that extends from or through a portion of the handle 104 into one of the apertures 354 of the blade 114.

The retention feature 556 may also facilitate movement of the blade 114 to one or more additional positions (e.g., a second position), whereby the blade 114 may extend out of the handle 104 and beyond the trial head 102 to cut the surface. The retention feature 556 may be configured to retract before or during impact of the blade 114 towards the trial head 102 such that the blade 114 may move along within the cavity 116 of the handle 104.

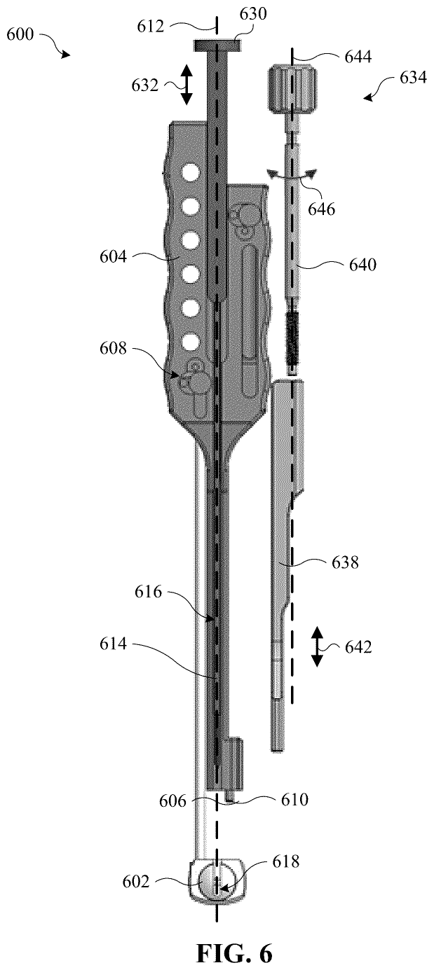

FIG. 6 illustrates an exploded view of a second example of a cervical disc instrument 600. The cervical disc instrument 600 may be similar to the cervical disc instrument 100 shown in FIGS. 1A and 1B unless otherwise stated. For example, the cervical disc instrument 600 may also be configured for use during TDR surgery to replace a damaged and/or deteriorated disc with a disc implant.

The cervical disc instrument 600 may include a trial head 602 that is removably coupled to a handle 604 of the cervical disc instrument 600. The trial head 602 may be similar to the trial head 102 described above, whereby the trial head 602 may be configured for insertion into a disc space. The trial head 602 may be removably coupled to the handle 604 by a trial head shaft 606 extending therebetween. By way of example, the trial head 602 may engage a latch mechanism 608 disposed in the handle 604 to removably couple the trial head 602 to the handle 604.

To ensure proper alignment between the trial head 602 and the handle 604, the cervical disc instrument 600 may also include an alignment post 610 that may extend from the handle 604 and into an alignment hole of the trial head 602. As such, the alignment post 610 may ensure proper alignment between the trial head 602 and the handle 604. Additionally, the alignment post 610 may also maintain proper orientation of the trial head 602 and the handle 604 with respect to one another along a longitudinal axis 612 of the cervical disc instrument 600.

The cervical disc instrument 600 may also include a blade 614. The blade 614 may be configured to cut a surface (e.g., a surface of bone, such as superior and/or inferior endplates at the operative level with respect to the disc space) that at least partially defines the disc space. The blade 614 may be at least partially disposed in a cavity 616 defined by the handle 604 and configured to cut the surface when the trial head 602 is located in the disc space.

By way of example, the blade 614 may extend and move along the longitudinal axis 612 of the cervical disc instrument 600. The trial head 602 and the blade 614 may be coaxial along the longitudinal axis 612 of the cervical disc instrument 600 and the trial head shaft 606 may be offset from the longitudinal axis 612 of the cervical disc instrument 600 such that the blade 614 may extend through a groove 618 of the trial head 602 to cut the surface (e.g., the bone surface). That is, the groove 618 of the trial head 602 may extend along the longitudinal axis 612 of the cervical disc instrument 600 to at least partially guide the blade 614 along the longitudinal axis 612 of the cervical disc instrument 600 during operation. As such, in one or more positions, the blade 614 may be disposed in the groove 618 of the trial head 602 so that the blade 614 moves along the groove 618 of the trial head 602. Thus, the cervical disc instrument 600 may facilitate trialing of the trial head 602 within the disc space while also allowing for cutting of the surface (e.g., the bone surface via the blade 614) when the trial head 602 is located in the disc space.

Operation of the cervical disc instrument 600 will now be described in further detail. To connect the trial head 602 to the handle 604, the alignment post 610 of the handle 604 may be aligned with an alignment hole of the trial head 602. Before, during, or after alignment of the alignment post 610, the trial head shaft 606 may be inserted into the latch mechanism 608 to couple the trial head 602 to the handle 604. The trial head 602 may be integrally (e.g., monolithically) formed with the trial head shaft 606 or the trial head shaft 606 may be coupled to the trial head 602 in a manner similar to the trial head 102 and the trial head shaft 106 of the cervical disc instrument 100 shown in FIGS. 1A and 1B. Based on such configuration, the trial head shaft 606—and thus the trial head 602—may be easily connected to, and disconnected from, the handle 604 via the latch mechanism 608.

Once the trial head 602 is coupled to the handle 604, the trial head 602 may be inserted into the disc space to determine a proper position (e.g., location and/or orientation) of the trial head 602 within the disc space. After positioning of the trial head 602, the blade 614 may be used to cut a surface of the bone surrounding the disc space. The blade 614 is not particularly limited to any type of cutting and may be used to perform any operation needed within the disc space.

By way of example, the blade 614 may include, or may be coupled to, an impact cap 630 that extends outward (e.g., positioned outside of) the handle 604. The impact cap 630 may be pressed (e.g., impacted) to move the blade 614 in a direction 632 towards and/or along the trial head 602 (e.g., along the groove 618 of the trial head 602). As a result, the blade 614 may cut one or more slots along the surface—or surfaces—that at least partially extend around the disc space, whereby a disc implant may be guided along the slots. Thus, the blade 614 may cut the slots in a precise location based upon the positioning of the trial head 602 within the disc space without removing the trial head 602 from its proper location.

To ensure that the slots or other cuts are completed at a desired depth, the cervical disc instrument 600 may also include a depth stop 634. The depth stop 634 may be removably coupled to the handle 604 or integrated into the handle 604. The depth stop 634 may be configured to define a cutting depth of the blade 614 with respect to the surface (e.g., the bone surface). The depth stop 634 may also be configured to prevent over-travel of the blade 614 (e.g., in the direction 632 towards the trial head 602), thereby preventing the blade 614 from cutting too deep and causing injury or permanent disability to the patient.

To facilitate such protection, the depth stop 634 may include a depth stop post 638 and a drive shaft 640 coupled to the depth stop post 638. The depth stop post 638 may be configured to contact the surface (e.g., the bone surface) or another surface in proximity to the surface being cut. The drive shaft 640 may be in communication with the depth stop post 638 to drive the depth stop post 638 in a direction 642 towards and away from the trial head 602. The depth stop post 138 may translate in the direction 642 beyond the trial head 602 to ensure contact with the surface. Additionally, movement of the depth stop post 638 in the direction 642 may dictate a cutting depth of the blade 614. That is, the depth stop post 638 may be driven in the direction 642 by the drive shaft 640 to a desired position, whereby the drive shaft 640 may maintain the desired position of the depth stop post 638 while cutting the surface with the blade 614. As such, a position of the depth stop post 138—and thus a cutting depth of the blade 614—may be adjusted based upon a given application.

It should be noted that the depth stop 634 may be positioned anywhere along the handle 604. For example, as shown in FIG. 6, the depth stop 634 may be offset from the longitudinal axis 612 of the cervical disc instrument 600 so that the depth stop 634 may be free of obstruction with the blade 614. The depth stop 634 may be positioned on an opposing side of the longitudinal axis 612 of the cervical disc instrument 600 compared to the trial head shaft 606, such as shown in FIG. 6, or the trial head shaft 606 and the depth stop 634 may be located on the same side of the longitudinal axis 612 of the cervical disc instrument 600.

To drive the depth stop post 638, the drive shaft 640 may be rotated about an axis of rotation 644 of the drive shaft 640 in a direction 646. The drive shaft 640 may be in communication with the depth stop post 638, such as via teeth or threading, such that rotation of the drive shaft 640 about the axis of rotation 644 of the drive shaft 640 may translate the depth stop post 638 in the direction 642 towards and away from the drive shaft 640. That is, rotation of the drive shaft 640 about the axis of rotation 644 of the drive shaft 640 may move the depth stop post 638 axially along, or parallel to, the axis of rotation 644 of the drive shaft 640.

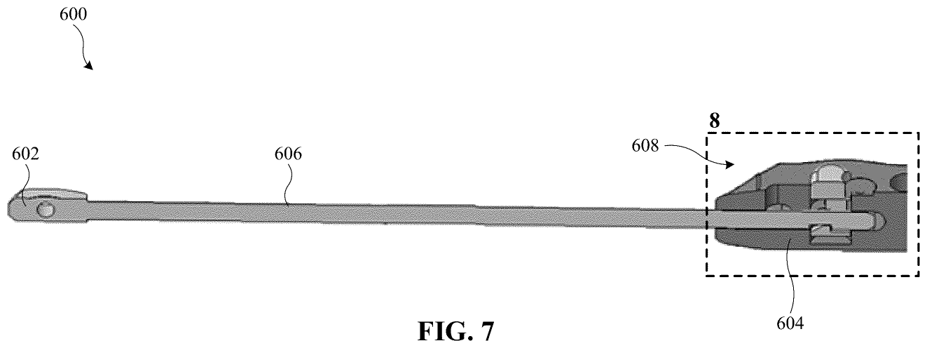

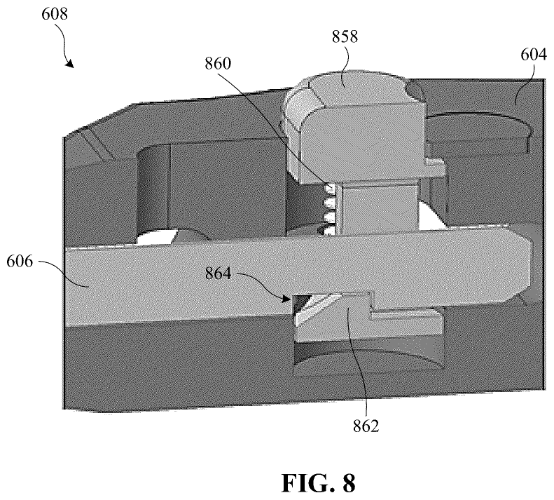

FIG. 7 is a partial cross-sectional view of the cervical disc instrument 600 shown in FIG. 6 to illustrate the latch mechanism 608. FIG. 8 is a close-up cross-section view of the latch mechanism 608 shown in FIG. 7. As described above, the trial head shaft 606 may be coupled to, or integrally formed with, the trial head 602 such that the trial head shaft 606 may be inserted into, or otherwise received by, the latch mechanism 608 disposed in the handle 604 of the cervical disc instrument 600.

To removably couple the trial head shaft 606 and thus the trial head 602 to the handle 604, the latch mechanism 608 may include a latch button 858 that is movable within the latch mechanism 608 (e.g., within a cavity of the latch mechanism 608). The latch button 858 may be configured to engage the trial head shaft 606 to couple the trial head shaft 606 to the handle 604. By way of example, a biasing member 860 may be configured to bias an engaging portion 862 of the latch button 858 towards a notch 864 of the trial head shaft 606 to maintain a position of the trial head shaft 606 when the trial head shaft 606 engages the latch mechanism 608. That is, the engaging portion 862 may be received by the notch 864 so that the engaging portion 862 may contact a wall of the notch 864 to prevent the trial head shaft 606 from disconnecting from the handle 604.

It should be noted that the engaging portion 862 and the notch 864 may be any complementary components between the latch button 858 and the trial head shaft 606 that maintain engagement therebetween. By way of example, as shown in FIG. 8, the engaging portion 862 may be a tooth or projection that is received by the notch 864, whereby the notch 864 may act as a female receiving portion for the engaging portion 862 (i.e., the male engaging portion). However, in certain configurations, the engaging portion 862 may be a female feature while the trial head shaft 606 may include a male feature similar to the engaging portion 862.

Additionally, the latch button 858 may be exposed along an outer surface of the handle 604 or may project through the outer surface of the handle 604. As a result, a user (e.g., a surgeon) may be able to compress the latch button 858 towards the trial head shaft 606. In doing so, the engaging portion 862 may disengage the notch 864 of the trial head shaft 606 due to compression of the biasing member 860, at which point the trial head shaft 606 may be removed from the latch mechanism 608. Once the trial head shaft 606 is removed and the user no longer compresses the latch button 858, the biasing member 860 may return the latch button 858 to its original position so that the latch button 858 may once again engage the trial head shaft 606 when the trial head shaft 606 is reinserted into the latch mechanism 608.

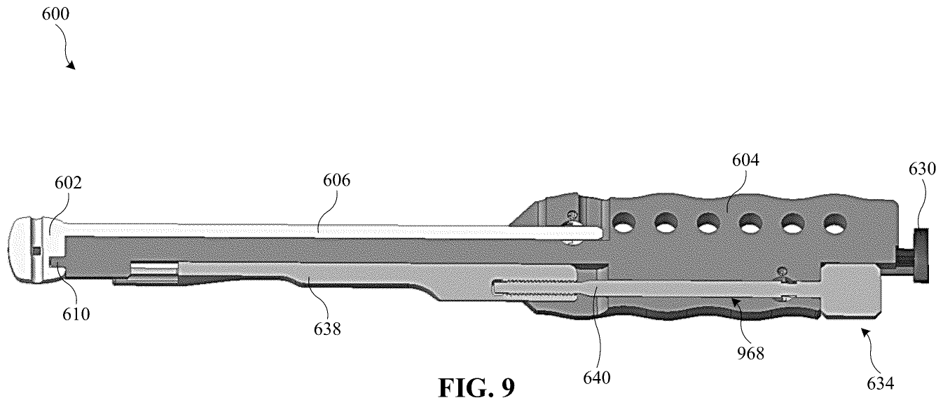

FIG. 9 is another cross-sectional view of the cervical disc instrument 600 to further illustrate a configuration of the depth stop 634. As discussed above, the depth stop 634 may include the depth stop post 638, which may be configured to contact the surface (e.g., the bone surface) that may be cut by the blade 614. The drive shaft 640 may be coupled to the depth stop post 638 such that, as shown in FIG. 6, rotation of the drive shaft 640 about the axis of rotation 644 of the drive shaft 640 moves the depth stop post 638 axially along the axis of rotation 644 of the drive shaft 640.

To maintain engagement between the depth stop 634 and the handle 604 of the cervical disc instrument 600, the drive shaft 640 may be engaged with a track 968 disposed in the handle 604. For example, the track 968 may be a secondary cavity defined by the handle 604 that may receive the drive shaft 640 therein. The drive shaft 640 may extend through the track 968 to couple to the depth stop post 638. For example, a first end of the drive shaft 640 may be extend beyond the track 968 and couple to the depth stop post 638 while an opposing second end of the drive shaft 640 may be exposed outside (e.g., external to) the track 968 such that the user (e.g., the surgeon) may drive (e.g., rotate) the depth stop 634.

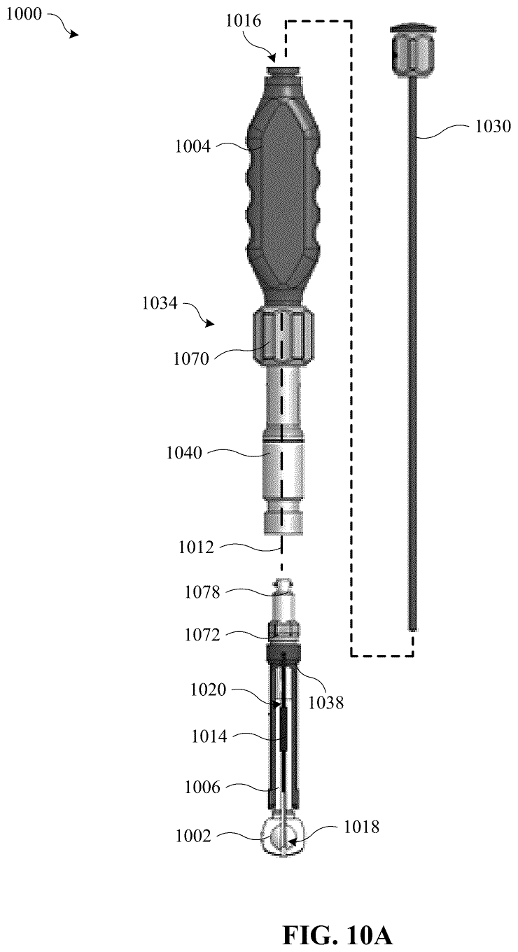

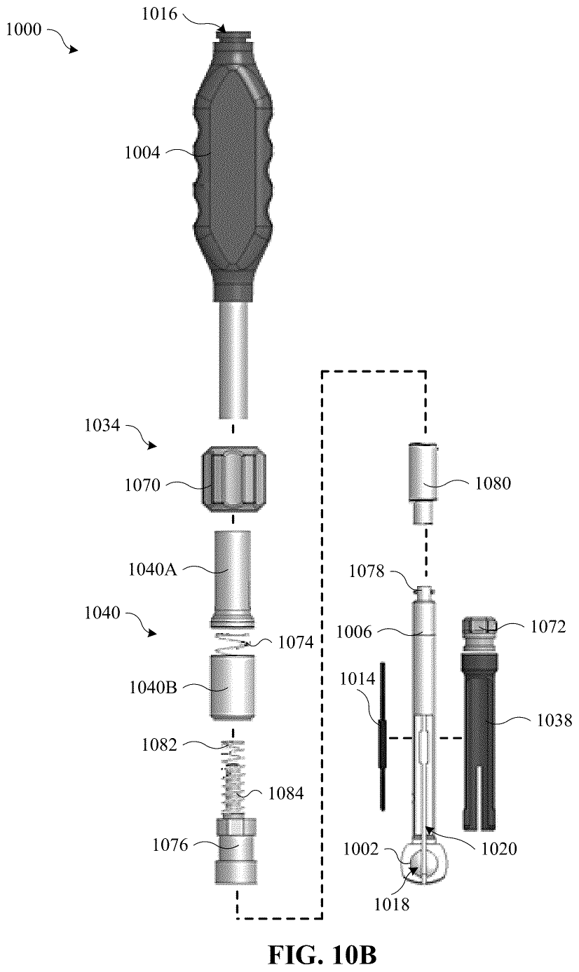

FIGS. 10A and 10B are exploded views of a third example of a cervical disc instrument 1000. The cervical disc instrument 1000 may be similar to the cervical disc instrument 100 and the cervical disc instrument 600 described above. For example, the cervical disc instrument 1000 may also be configured for use during TDR surgery to replace a damaged and/or deteriorated disc with a disc implant.

The cervical disc instrument 1000 may include a trial head 1002 that is removably coupled to a handle 1004 of the cervical disc instrument 1000. The trial head 1002 may be similar to the trial head 102 and/or the trial head 602 described above, whereby the trial head 1002 may be configured for insertion into a disc space. The trial head 1002 may be removably coupled to the handle 1004, either directly or indirectly, by a trial head shaft 1006 extending therebetween. By way of example, the trial head 1002 may be removably coupled to the handle 1004 such that the handle 1004 and the trial head 1002 may be substantially coaxial along a longitudinal axis 1012 of the cervical disc instrument 1000. It should also be noted that the handle 1004 may define a cavity 1016 therein that may extend through all or a portion of the handle 1004 along the longitudinal axis 1012 of the cervical disc instrument 1000.

The cervical disc instrument 1000 may also include a blade 1014. The blade 1014 may be configured to cut a surface (e.g., a surface of bone, such as superior and/or inferior endplates at the operative level with respect to the disc space) that at least partially defines the disc space. The blade 1014 may be at least partially disposed in a groove 1018 of the trial head 1002 and/or a groove 1020 of the trial head shaft 1006. As shown in FIG. 10A, the groove 1018 and the groove 1020 may be coaxial along the longitudinal axis 1012 of the cervical disc instrument 1000 and may extend along the longitudinal axis 1012 of the cervical disc instrument 1000 to allow the blade 1014 to move along the groove 1018 of the trial head 1002 and the groove 1020 of the trial head shaft 1006.

As discussed above, the handle 1004 may define a cavity 1016 therein. The cervical disc instrument 1000 may further include a rod 1030 that extends through the cavity 1016 to contact the blade 1014 disposed in the groove 1020 of the trial head shaft 1006. The rod 1030 may be movable within the cavity 1016 (e.g., via the surgeon manually moving the rod 1030) to move the blade 1014 along the groove 1020 of the trial head shaft 1006 and/or the groove 1018 of the trial head 1002. For example, the rod 1030 may move along the longitudinal axis 1012 of the cervical disc instrument 1000 within the cavity 1016 to contact an end of the blade 1014 to move the blade 1014 along the groove 1020 of the trial head shaft 1006 to extend the blade 1014 beyond the trial head 1002 to contact the surface to be cut. After cutting, the rod 1030 may be removed from the cavity 1016 or otherwise retracted from engagement with the blade 1014 so that the blade 1014 may also be retracted via a retraction mechanism of the cervical disc instrument 1000, such as a biasing member (e.g., a spring).

The cervical disc instrument 1000 may also include a depth stop 1034. The depth stop 1034 may be configured to define a cutting depth of the blade 1014 with respect to the surface (e.g., the bone surface). As shown in FIG. 10A, the depth stop 1034, the handle 1004, the trial head 1002, and the blade 1014 may be coaxial along the longitudinal axis 1012 of the cervical disc instrument 1000, thereby providing a more optimized packaging space.

The depth stop 1034 may include a depth stop post 1038 and a drive shaft 1040 in communication with the depth stop post 1038. The drive shaft 1040 may include a first portion 1040A and a second portion 1040B coupled to and/or in communication with the first portion 1040A. The depth stop post 1038 may be configured to contact the surface (e.g., the bone surface) or another surface in proximity to the surface being cut. The drive shaft 1040 may be in communication with the depth stop post 1038 to drive the depth stop post 1038 towards and away from the trial head 1002. For example, a knob 1070 of the depth stop 1034 may be rotated by the user (e.g., the surgeon), such as in a direction of rotation about the longitudinal axis 1012 of the cervical disc instrument 1000, whereby rotation of the knob 1070 may translate the drive shaft 1040 towards the depth stop post 1038. As a result, the drive shaft 1040 may translate the depth stop post 1038 towards and/or beyond the trial head 1002.

To provide further adjustability, the depth stop 1034 may also include a depth adjustment nut 1072, which may be threaded or otherwise coupled to a portion of the cervical disc instrument 1000 to adjust a depth of the depth stop post 1038. That is, the depth adjustment nut 1072 may determine how far the depth stop post 1038 may extend beyond a terminal end of the trial head 1002. To adjust the depth of the depth stop post 1038, the depth adjustment nut 1072 may be rotated about the longitudinal axis 1012 of the cervical disc instrument 1000 to define a starting position of the depth stop post 1038 prior to engaging the depth stop 1034 via the knob 1070.

To further illustrate the depth stop 1034, FIG. 10B provides a further exploded view of the cervical disc instrument 1000, and in particular, an exploded view of the depth stop 1034. As shown in FIG. 10B, knob 1070 may be coupled to, and in communication with, the first portion 1040A of the drive shaft 1040. Additionally, the first portion 1040A of the drive shaft 1040 may be coupled to and/or in communication with the second portion 1040B of the drive shaft 1040, whereby a drive spring 1074 may be disposed therebetween. For example, the drive spring 1074 may be disposed within a cavity defined by both the first portion 1040A and the second portion 1040B of the drive shaft 1040.

The drive spring 1074 may be in communication with a depth stop drive 1076 of the drive shaft 1040 to bias the depth stop drive 1076 towards the depth adjustment nut 1072, thereby biasing the depth adjustment nut 1072 towards (e.g., against) the depth stop post 1038. As a result, the drive spring 1074 may bias the depth stop 1034 towards the depth stop post 1038 to ensure contact between the components of the depth stop 1034. Thus, the drive spring 1074 may ensure that rotation of the knob 1070 is translated through the depth stop 1034 (e.g., through the drive shaft 1040, the depth stop drive 1076, and the depth adjustment nut 1072) to translate the depth stop post 1038 along the longitudinal axis 1012 of the cervical disc instrument 1000.

As discussed above, the trial head shaft 1006 may be removably coupled to the handle 1004 to facilitate easy connection and disconnect between the trial head shaft 1006 and the handle 1004. To facilitate such removably coupling, the trial head shaft 1006 may be connected to the handle 1004 by a bayonet 1078. By way of example, the trial head shaft 1006 may include the bayonet 1078, whereby the bayonet 1078 may be received by a bayonet sleeve 1080 to removably couple the trial head 1002 to the handle 1004. As shown in FIGS. 10A and 10B, the bayonet sleeve 1080 may be disposed in the drive shaft 1040 and/or the depth stop drive 1076 to decrease an overall packaging length of the cervical disc instrument 1000.

The bayonet 1078 may include one or more studs that are configured to engage the bayonet sleeve 1080 to couple the trial head shaft 1006 to the handle 1004. By way of example, to connect the bayonet 1078 to the bayonet sleeve 1080, the bayonet 1078 may be inserted into the bayonet sleeve 1080 and rotated with respect to the bayonet 1078 (e.g., along a longitudinal axis of the bayonet sleeve 1080) such that the studs of the bayonet 1078 may be received by a receiving portion of the bayonet sleeve 1080. Once the studs are aligned with the receiving portion of the bayonet sleeve 1080, a bayonet biasing member 1082 disposed around a plunger 1084 that extends from the depth stop drive 1076 may bias the studs into the receiving portion of the bayonet sleeve 1080, thereby maintaining connection between the trial head shaft 1006 and the handle 1004.

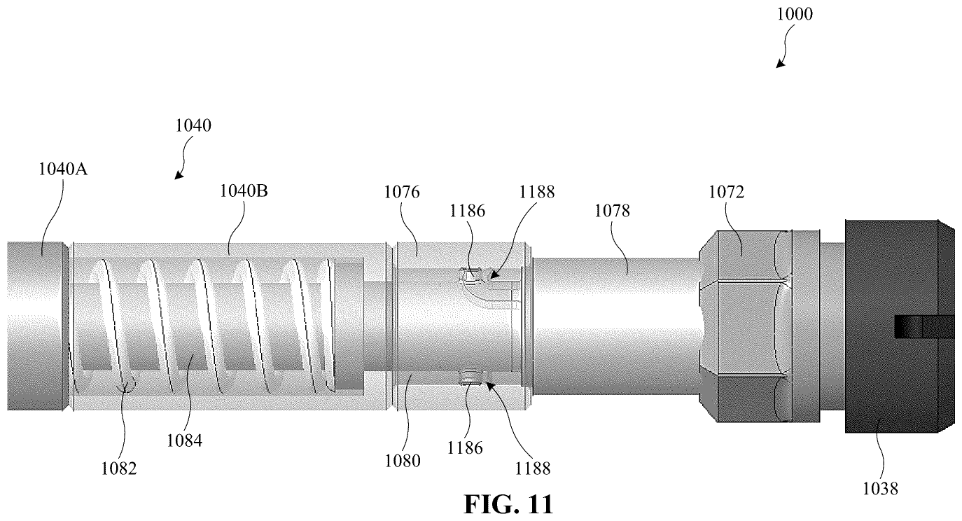

FIG. 11 illustrates a close-up view of the connection between trial head shaft 1006 and the handle 1004. In particular, FIG. 11 illustrates a close-up view of the connection between the bayonet 1078 and the bayonet sleeve 1080. As discussed above, the bayonet 1078 may include one or more studs 1186 that project from (e.g., extend radially away from) the bayonet 1078. To couple the trial head shaft 1006 to the handle 1004, the bayonet 1078 may be inserted into the bayonet sleeve 1080—which may be at least partially disposed within the depth stop drive 1076 and/or the drive shaft 1040—and rotated with respected to the bayonet sleeve 1080 so that the studs 1186 may extend into slots 1188 of the bayonet sleeve 1080. The slots 1188 may be any groove, cutout, or channel of the bayonet sleeve 1080 that may be complementary in shape to the studs 1186 (e.g., pins, projects, teeth, etc.) so that the slots 1188 may receive and retain the studs 1186.

To maintain connection between the bayonet 1078 and the bayonet sleeve 1080, the bayonet biasing member 1082 disposed around the plunger 1084 may bias the studs 1186 towards and into the slots 1188. For example, the bayonet biasing member 1082 may be a spring that is located within the second portion 1040B of the drive shaft 1040 and compressed (e.g., pre-loaded) by an internal surface of the drive shaft 1040 so that the bayonet biasing member 1082 may bias the studs 1186 towards and into the slots 1188.

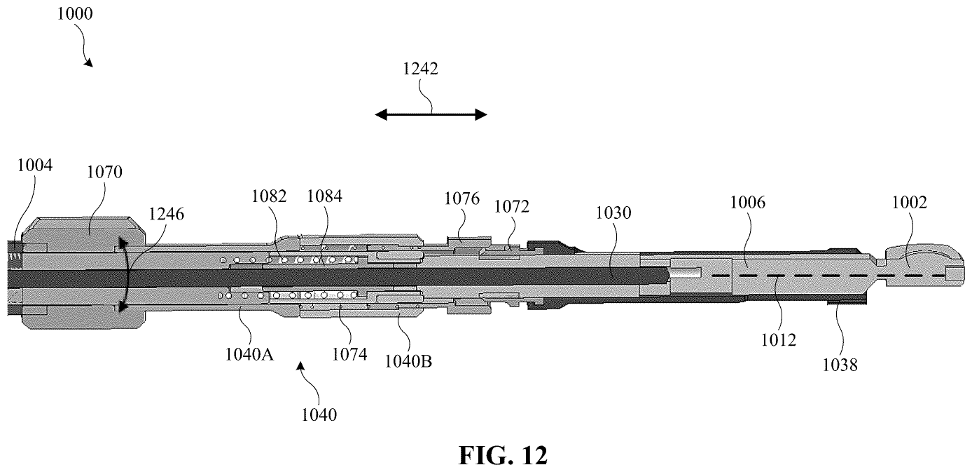

FIG. 12 illustrates a cross-sectional view of the cervical disc instrument 1000 shown in FIGS. 10A-11. The blade 1014 and other details, such as threading of one or more components, is removed for clarity and simplicity. As discussed above, the cervical disc instrument 1000 may include the handle 1004 that is removably coupled to the trial head 1002 by the trial head shaft 1006. In particular, the trial head shaft 1006 may include the bayonet 1078, which may be biased towards the bayonet sleeve 1080 via the bayonet biasing member 1082 to maintain engagement between the trial head shaft 1006 and the handle 1004.

To adjust a depth in which the blade 1014 may cut through a surface, the cervical disc instrument 1000 may also include the depth stop 1034. The depth stop 1034 may be configured to move the depth stop post 1038 in a direction 1242 along the longitudinal axis 1012 of the cervical disc instrument 1000 towards and/or beyond the trial head 1002 to prevent over-travel of the blade 1014 into the surface being cut. To facilitate such movement of the depth stop post 1038, the depth stop 1034 may include the knob 1070. The knob may be rotated in a direction 1246 about the longitudinal axis 1012 of the cervical disc instrument 1000, which may in turn drive the drive shaft 1040 and the depth stop drive 1076 towards and into the depth adjustment nut 1072 in the direction 1242. The depth stop drive 1076 may then in turn drive the depth stop post 1038 in the direction 1242 towards and/or beyond the trial head 1002.

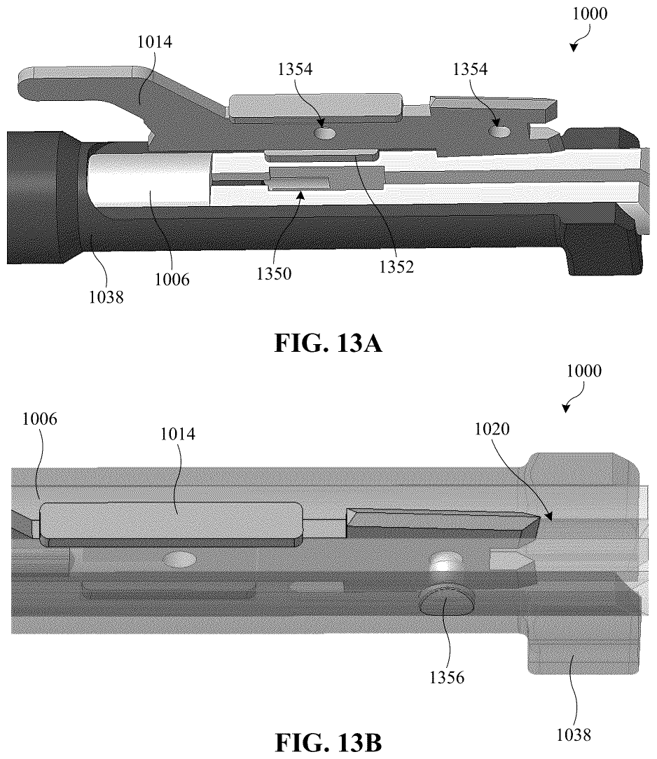

FIG. 13A illustrates a close-up view of the cervical disc instrument 1000 prior to insertion of the blade 1014 into the groove 1020 of the trial head shaft 1006. FIG. 13B illustrates another close-up view of the cervical disc instrument 1000 after insertion of the blade 1014 into the groove 1020 of the trial head shaft 1006. As discussed above, the blade 1014 may be disposed in the groove 1020 of the trial head shaft 1006 and configured to move along the groove 1020 of the trial head shaft 1006.

The blade 1014 may be configured for insertion into the groove 1020 of the trial head shaft 1006 through a keyed opening 1350 defined by the groove 1020 of the trial head shaft 1006. The keyed opening 1350 may be keyed (e.g., shaped) in a complementary manner to a keying feature 1352 of the blade 1014. As a result, the keyed opening 1350 and the keying feature 1352 may ensure that the blade 1014 is inserted into the groove 1020 of the trial head shaft 1006 in a proper location and with a proper orientation. Moreover, the keying feature 1352 of the blade 1014 may retain the blade 1014 within the groove 1020 of the trial head 1002. For example, the groove 1020 may be defined at least partially by one or more flanges and the keying feature 1352 of the blade 1014 may engage the one or more flanges to prevent the blade 1014 from being removed from the groove 1020.

The blade 1014 may also include one or more apertures 1354. The apertures 1354 may be positioned anywhere along the blade 1014. For example, the apertures 1354 may be disposed along the blade 1014 such that the apertures 1354 are positioned within the groove 1020 of the trial head shaft 1006 when the blade 1014 is inserted into the groove 1020. The apertures 1354 may be positioned and configured to receive or otherwise engage a retention feature 1356 of the trial head shaft 1006. That is, the retention feature 1356 may retain the blade 1014 in an initial position within the groove 1020 of the trial head shaft 1006.

By way of example, the retention feature 1356 may be a pin, spring, plunger, or other type of mechanical feature that may be received by at least one of the apertures 1354 to maintain a position of the blade 1014 along the longitudinal axis 1012 of the cervical disc instrument 1000. The retention feature 1356 may then be released in any desired manner to allow for movement of the blade 1014 in the direction 1242 shown in FIG. 12 along the longitudinal axis 1012 of the cervical disc instrument 1000.

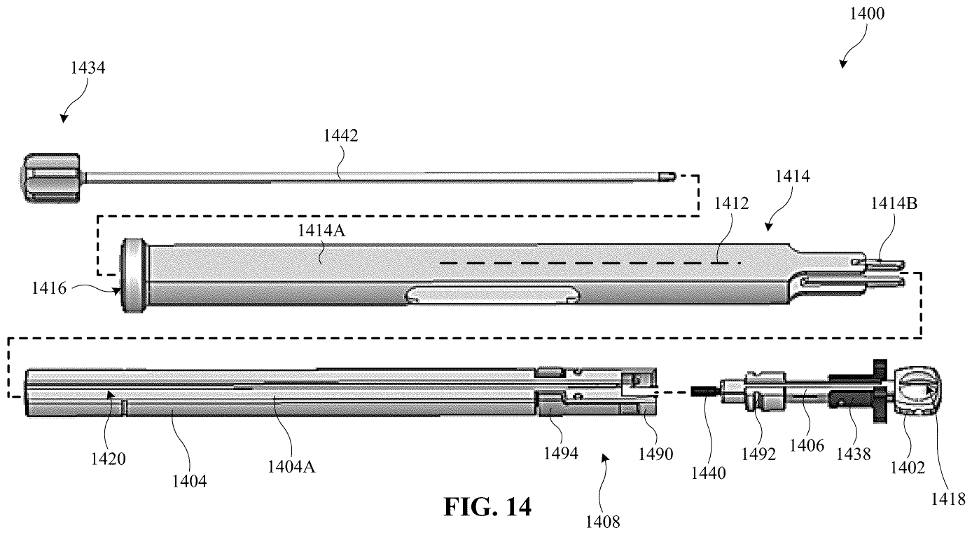

FIG. 14 is an exploded view of a fourth example of a cervical disc instrument 1400. The cervical disc instrument 1400 may be similar to the cervical disc instrument 100, the cervical disc instrument 600, and the cervical disc instrument 1000 described above. For example, the cervical disc instrument 1400 may also be configured for use during TDR surgery to replace a damaged and/or deteriorated disc with a disc implant.

The cervical disc instrument 1400 may include a trial head 1402 that is removably coupled to a handle 1404 of the cervical disc instrument 1400. The trial head 1402 may be similar to the trial head 102, the trial head 602, or the trial head 1002 described above, whereby the trial head 1402 may be configured for insertion into a disc space for trialing. The trial head 1402 may be removably coupled to the handle 1404, either directly or indirectly, by a trial head shaft 1406 extending therebetween. By way of example, the trial head 1402 may be removably coupled to the handle 1404 via a latch mechanism 1408 such that the handle 1404 and the trial head 1402 may be substantially coaxial along a longitudinal axis 1412 of the cervical disc instrument 1400 when the cervical disc instrument 1400 is assembled.

The cervical disc instrument 1400 may also include a blade component 1414 that includes a shaft portion 1414A and a blade portion 1414B, whereby the shaft portion 1414A may define a cavity 1416 therein. The blade component 1414 may be movable coupled to the handle 1404 and the trial head 1002, whereby a groove 1418 of the trial head 1402 and a groove 1420 of the handle 1404 may guide the blade component 1414. By way of example, the groove 1420 may be located on an exterior surface of the handle 1404, such as the exterior surface 1404A. The groove 1420 may extend along the longitudinal axis 1412 of the cervical disc instrument 1400 to guide the blade component 1414 along the longitudinal axis 1412 to cut a surface (e.g., a bone surface). That is, the blade component 1414 may be slidably coupled to the handle 1404. For example, the groove 1418 of the trial head 1402 may be is configured to receive the blade portion 1414B of the blade component 1414 to guide the blade component 1414 along the groove 1418. The groove 1418 of the trial head 1402 may be coaxial with the groove 1420 of the handle 1404.

To facilitate engagement between the blade component 1414 and the handle 1404, the shaft portion 1414A may be configured to at least partially enclose the handle 1404. That is, the shaft portion 1414A may define the cavity 1416 and the handle 1404 may be inserted into the cavity 1416. The blade portion 1414B may extend from the shaft portion 1414A. The blade portion 1414B may also extend at least partially into the cavity 1416 of the shaft portion 1414A to engage the groove 1420 of the handle 1404 and guide the blade component 1414 along the groove 1420. Thus, the blade component 1414 may move along the groove 1420 to move along the longitudinal axis 1412 of the cervical disc instrument 1400. For example, the blade component 1414 may extend beyond the trial head 1402 when the trial head 1402 is inserted into the disc space to cut a surface that at least partially defines the disc space.

The cervical disc instrument 1400 may also include a depth stop 1434. The depth stop 1434 may be configured to prevent over-travel of the blade component 1414 to thereby prevent cutting into the surface deeper than desired. The depth stop 1434 may be positioned anywhere along the cervical disc instrument 1400. For example, the depth stop 1434 may be coaxial with the handle 1404, the trial head 1402, and the blade component 1414.

The depth stop 1434 may include a depth stop post 1438. The depth stop post 1438 may be positioned between the handle 1404 and the trial head 1402. A drive shaft 1440 may be coupled to the depth stop post 1438 and configured to drive movement of the depth stop post 1438. By way of example, the drive shaft 1440 may be configured to rotated about an axis of rotation of the drive shaft 1440, such as the longitudinal axis 1412 of the cervical disc instrument 1400, to move (e.g., translate) the depth stop post 1438 axially along the axis of rotation (e.g., the longitudinal axis 1412 of the cervical disc instrument 1400).

It is envisioned that the drive shaft 1440 may be directly or indirectly driven. For example, as shown in FIG. 14, a depth stop shaft 1442 may be coupled (e.g., threadably engaged) to the drive shaft 1440 such that rotation of the depth stop shaft 1442 may rotate the drive shaft 1440. In certain configurations, the drive shaft 1440 and the depth stop shaft 1442 may be integrally formed with one another.

As discussed above, the trial head 1402 may be removably coupled to the handle 1404 by the latch mechanism 1408. By way of example, the latch mechanism 1408 may include one or more latches 1490 formed with, or coupled to, the handle 1404. The latches 1490 may be configured to engage one or more fingers 1492 of the trial head 1402. For example, the latches 1490 may include a button 1494 that is configured to be compressed to thereby disengage the latches 1490 from the trial head 1002 (e.g., from the fingers 1492) to disconnect the trial head 1402 from the handle 1404. Thus, connection and disconnect between the trial head 1402 and the handle 1404 may be done easily and quickly by the user (e.g., the surgeon).

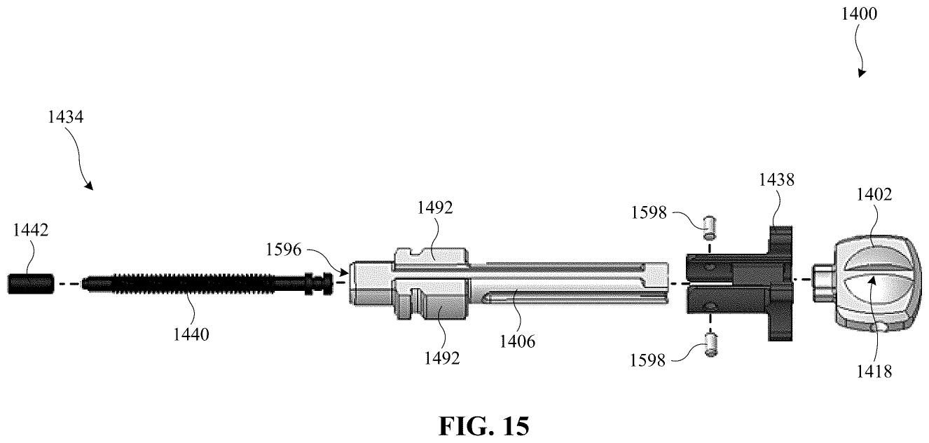

FIG. 15 illustrates another exploded view of the cervical disc instrument 1400 shown in FIG. 14. As discussed above, the cervical disc instrument 1400 may include the trial head 1402, whereby the trial head 1402 may be removably coupled to the handle 1404 via the trial head shaft 1406. The trial head 1402 may be coupled to the handle 1404 via the latch mechanism 1408. For example, a pair of the latches 1490 may be formed with, or coupled to, the handle 1404 and configured to engage respective ones of the fingers 1492 of the trial head 1402. As shown in FIG. 15, the trial head 1402 may include, or be coupled to, the trial head shaft 1406 and the fingers 1492 may be located on the trial head shaft 1406. The pair of the latches 1490 may be configured for positioning on opposing sides of the trial head shaft 1406 to engage the respective ones of the fingers 1492.

The cervical disc instrument 1400 may also include the depth stop 1434. As discussed above, the depth stop 1434 may include the drive shaft 1440 and the depth stop shaft 1442. The drive shaft 1440 may be driven (e.g., rotated) based upon rotation of the depth stop shaft 1442, which may thereby drive the depth stop post 1438 towards and/or way from the trial head 1402.

The drive shaft 1440 may extend through an aperture 1596 defined by the trial head shaft 1406. The drive shaft 1440 may extend through the trial head shaft 1406 so that the depth stop post 1438 may be coupled to the drive shaft 1440 via depth stop pins 1598. The depth stop pins 1598 may also guide the depth stop post 1438 when the depth stop post 1438 is driven by the drive shaft 1440. For example, the depth stop pins 1598 may be guided along one or more channels, slots, grooves, cutouts, or a combination thereof defined by the trial head shaft 1406 to ensure translational movement of the depth stop post 1438 along the longitudinal axis 1412 of the cervical disc instrument 1400.

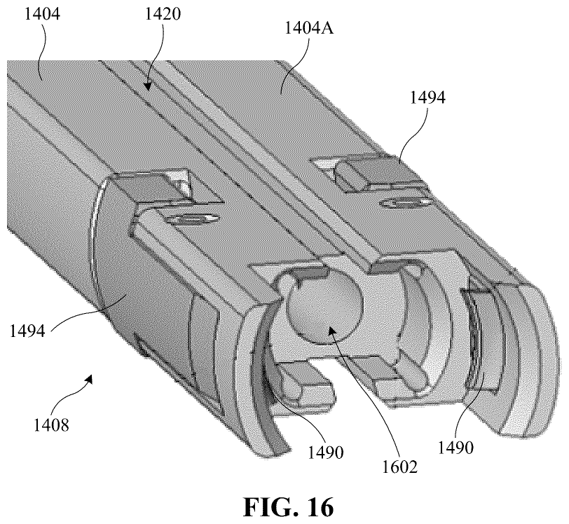

FIG. 16 illustrates a close-up perspective view of the handle 1404 of the cervical disc instrument 1400 to better illustrate the latch mechanism 1408. As discussed above, the latch mechanism 1408 may be configured to engage the fingers 1492 of the trial head shaft 1406 to removably couple the trial head 1402 (e.g., via the trial head shaft 1406) to the handle 1404. The latch mechanism 1408 may include the latches 1490 which may engage the fingers 1492 of the trial head shaft 1406. Each of the latches 1490 may include a respective one of the buttons 1494, which may be compressed to disengage the latches 1490 from the fingers 1492.

It should also be noted that the handle 1404 and the latch mechanism 1408 may accommodate operation of the depth stop 1434. For example, at least a portion of the drive shaft 1440 of the depth stop 1434 may be received by an aperture 1602 defined by the handle 1404. The drive shaft 1440 may thus be coupled to the depth stop shaft 1442 within the aperture 1602 of the handle 1404 free of obstruction to operation of the latch mechanism 1408.

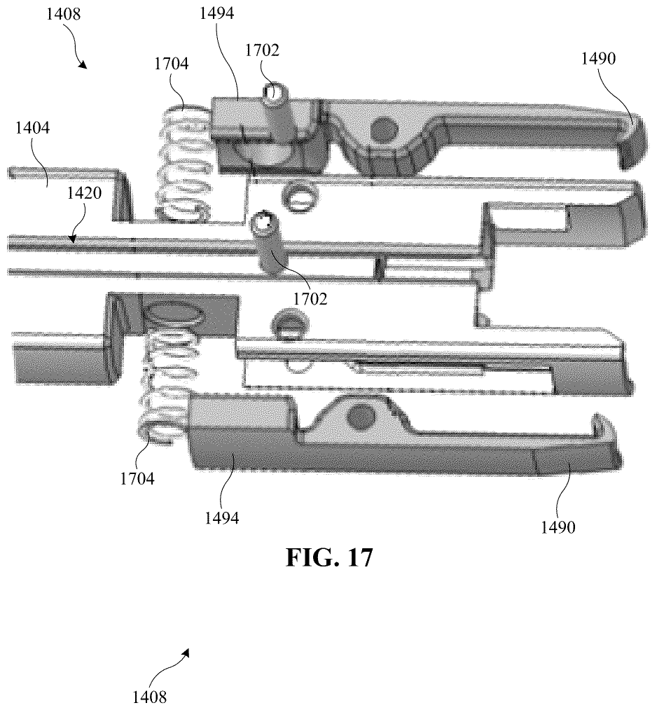

FIG. 17 illustrates an exploded view of the latch mechanism 1408 of the cervical disc instrument 1400. As shown in FIG. 17, the latches 1490 may be movably coupled to the handle 1404 via latch pins 1702. The latches 1490 may be configured to pivot with respect to the handle 1404 about an axis of rotation defined by respective ones of the latch pins 1702 so that the latches 1490 may pivot towards and away from the handle 1404. The latches 1490 may be configured for biasing towards the fingers 1492 of the trial head shaft 1406 to maintain engagement between the latches 1490 and respective ones of the fingers 1492. For example, each of the latches 1490 may biased towards the fingers 1492 via a latch biasing element 1704, whereby the latch biasing element 1704 may be positioned between the latch 1490 and the handle 1404.

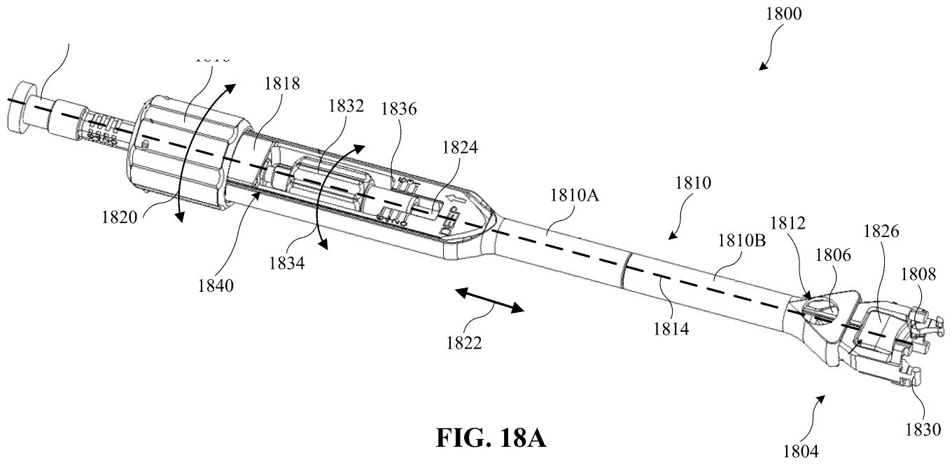

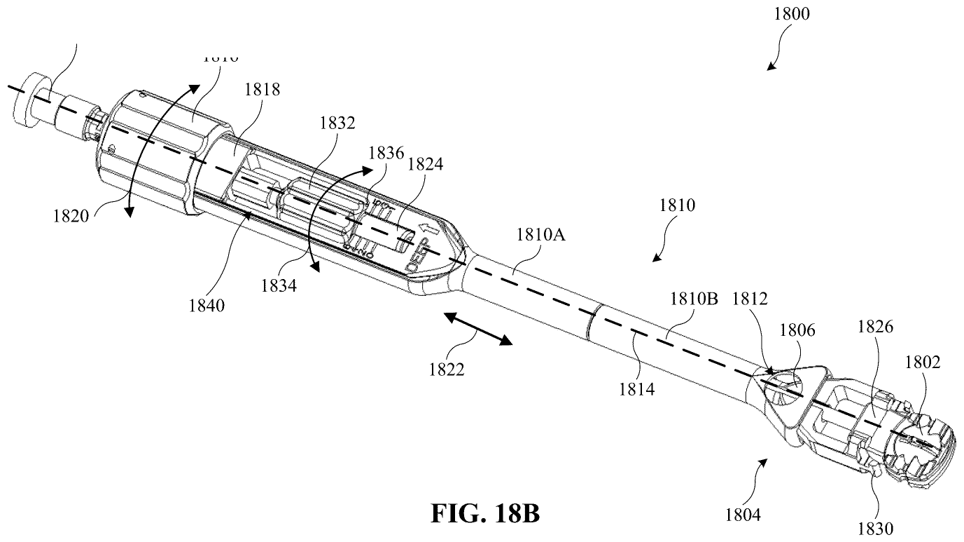

FIG. 18A is a perspective view of a fifth example of a cervical disc instrument 1800. While the cervical disc instruments 100, 600, 1000, and 1400 describe above may be configured for trialing of a trial head (e.g., the trial head 102 of the cervical disc instrument 100) within a disc space and/or cutting a surface defining the disc space, the cervical disc instrument 1800 may be configured to insert a disc implant 1802 into the disc space. As shown in FIGS. 18A and 18B, the disc implant 1802 may be removably coupled to the cervical disc instrument 1800 for insertion of the disc implant 1802 into and/or removal of the disc implant 1802 from the disc space.

The cervical disc instrument 1800 may include a jaw component 1804. The jaw component 1804 may include a jaw shaft 1806 and jaws 1808 extending from the jaw shaft 1806. The jaw component 1804 may include one or more of the jaws 1808. The jaws 1808 may be configured to grasp the disc implant 1802. By way of example, the jaws 1808 may be teeth or other engaging portions that engage a portion (e.g., a receiving portion) of the disc implant 1802 to couple the disc implant 1802 to the cervical disc instrument 1800. As discussed in further detail below, the jaws 1808 may be configured to move (e.g., pivot) with respect to a portion of the cervical disc instrument 1800 to couple to, and uncouple from, the disc implant 1802.

The cervical disc instrument 1800 may also include a sheath 1810. For example, the cervical disc instrument 1800 may include a two-piece sheath that may include a first portion 1810A and a second portion 1810B coupled to, or in communication with, the first portion 1810A. However, the sheath 1810 in certain configurations may also be a single-piece sheath. Additionally, the sheath 1810 may define a cavity 1812 therein, whereby the jaw shaft 1806 may be positioned within the cavity 1812 of the sheath 1810. The cavity 1812 may be defined by the first portion 1810A and/or the second portion 1810B of the sheath 1810. For example, the cavity 1812 of the sheath 1810 may extend through all or a portion of a length of the sheath 1810 that extends along a longitudinal axis 1814 of the cervical disc instrument 1800.

The sheath 1810 may be configured to translate along the longitudinal axis 1814 of the cervical disc instrument 1800 to actuate (e.g., move, rotate, etc.) the jaws 1808 to couple to, or uncouple from, the disc implant 1802. By way of example, the sheath 1810 may be configured to translate along the longitudinal axis 1814 of the cervical disc instrument 1800 to move from a first position, in which the sheath 1810 is spaced apart from the jaws 1808, to a second position, in which the sheath 1810 contacts the jaws 1808 to move the jaws 1808 towards one another to grasp the disc implant 1802. The sheath 1810 (e.g., the second portion 1810B of the sheath 1810) may contact an outer surface of the jaws 1808 or any other portion of the jaws 1808 to move the jaws 1808 toward one another to grasp the disc implant 1802. It should also be noted that one more positions of the jaws 1808 between the first position and the second position may also be possible based upon translation of the sheath 1810 along the longitudinal axis 1814 of the cervical disc instrument 1800.

To drive movement of the sheath 1810, a sheath knob 1816 may be coupled to the sheath 1810. For example, as shown in FIGS. 18A and 18B, the sheath knob 1816 may be movably coupled (e.g., threadably engaged) to the first portion 1810A of the sheath 1810. The sheath knob 1816 may also be coupled to a body 1818 to maintain a lateral and longitudinal position of the sheath knob 1816 with respect to the longitudinal axis 1814 of the cervical disc instrument 1800. As a result, rotation of the sheath knob 1816 in a direction 1820 about the longitudinal axis 1814 of the cervical disc instrument 1800 may translate the sheath 1810 along the longitudinal axis 1814 of the cervical disc instrument 1800 in a direction 1822 to drive opening and/or closing of the jaw 1808.

Based on operation of the sheath 1810 as described above, the disc implant 1802 may be coupled to the cervical disc instrument 1800 to insert the disc implant 1802 in the disc space. Once the disc implant 1802 is properly positioned in the disc space, the cervical disc instrument 1800 may also be configured to impact the disc implant 1802 to properly seed the disc implant 1802 in the disc space. To impact the disc implant 1802, the cervical disc instrument 1800 may include a drive shaft 1824 and a tamp 1826 coupled to the drive shaft 1824.

The drive shaft 1824 may extend along the longitudinal axis 1814 of the cervical disc instrument 1800 and may be configured to move the tamp 1826 towards and away from the disc implant 1802 coupled to the jaws 1808, such as in the direction 1822 along the longitudinal axis 1814 of the cervical disc instrument 1800.

By way of example, the drive shaft 1824 may extend through both the cavity 1812 of the sheath 1810 and a cavity defined by the jaw shaft 1806 to couple to the tamp 1826, whereby the tamp 1826 may be positioned between the jaws 1808 of the jaw component 1804. An impact cap 1828 may be in communication with and/or coupled to the drive shaft 1824. Impact of the impact cap 1828 may translate the drive shaft 1824 and thus the tamp 1826 in the direction 1822 along the longitudinal axis 1814 of the cervical disc instrument 1800.

To illustrate operation of the tamp 1826, FIGS. 18A and 18B will be described in further detail. FIG. 18A illustrates a first position of the tamp 1826 in which the tamp 1826 is in a retracted state. In the retracted state, the tamp 1826 may be located between the jaws 1808 and recessed from the jaws 1808 along the longitudinal axis 1814 of the cervical disc instrument 1800 so that the disc implant 1802 may be received by, and coupled to the jaws 1808 (e.g., using the sheath 1810 as described above). As such, the cervical disc instrument 1800 in combination with the disc implant 1802 may be considered a cervical disc replacement system.

The tamp 1826 may move from the first position (e.g., retracted state) to a second position in which the tamp 1826 is in an extended state, such as the extended state shown in FIG. 18B. As shown in FIG. 18B, in the extended state, the tamp 1826 may extend (e.g., translate) in the direction 1822 along the longitudinal axis 1814 between the jaws 1808 expand the jaws 1808 and/or extend beyond the jaws 1808 into the disc space. That is, the tamp 1826 may expand the jaws 1808 when the jaws 1808 are grasping the disc implant 1802 to release the disc implant 1802 in a desired position within the disc space. As discussed above, such operation of the tamp 1826 may be facilitated by impacting the impact cap 1828, which may in turn impact the disc implant 1802 in the disc space via the tamp 1826.

During insertion of the disc implant 1802, a contact feature 1830 of the jaw component 1804 may be configured to contact a surface (e.g., a bone surface) located adjacent to the disc space. Such contact between the contact feature 1830 and the surface may mitigate or prevent over-insertion of the disc implant 1802 into the disc space. The contact feature 1830 may be and size and/or shape. As described further below, the contact feature 1830 may include one or more lobes that may be configured to contact the surface to prevent over-insertion of the disc implant 1802 into the disc space.

To further prevent over-insertion of the disc implant 1802, the cervical disc instrument may also include a depth stop. The depth stop may be any feature or mechanism that may physically prevent over-insertion of the disc implant 1802 that may be caused by extension of the drive shaft 1824 into the disc space. By way of example, the depth may be or may include a depth stop knob 1832. The depth stop knob 1832 may be engaged (e.g., threadably engaged) to the drive shaft 1824. As such, a position of the depth stop knob 1832 axially along the drive shaft 1824 (e.g., along the longitudinal axis 1814 of the cervical disc instrument 1800) may be adjusted based upon rotating the depth stop knob 1832 in a direction 1834 about the longitudinal axis 1814 of the cervical disc instrument 1800.

To prevent over-travel, the depth stop knob 1832 may be configured to contact a lip 1836. The lip 1836 may be formed or part of the sheath 1810 and/or the body 1818. By way of example and as shown in FIGS. 18A and 18B, the depth stop knob 1832 may be positioned in a window 1840 defined by the body 1818 so that, when the drive shaft 1824 moves toward the jaws 1808 in the direction 1822, the depth stop knob 1832 may contact the lip 1836, whereby the lip 1836 may be formed as part of the body 1818 and may at least partially define the window 1840.

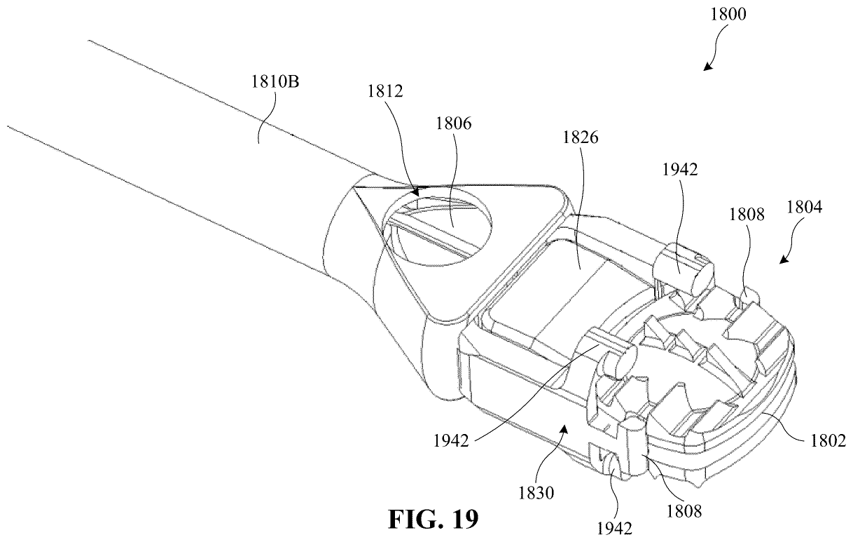

FIG. 19 is a close-up perspective view of the cervical disc instrument 1800 in which the disc implant 1802 is coupled to the jaws 1808 and the tamp 1826 is in a retracted state. In particular, FIG. 19 further illustrates the contact feature 1830 of the jaw component 1804. As discussed above, the contact feature 1830 may be configured to contact a surface (e.g., a bone surface) located adjacent to the disc space to prevent over-insertion of the disc implant 1802 into the disc space. By way of example, the contact feature 1830 may include one or more lobes 1942 positioned adjacent to the jaws 1808. As shown in FIG. 19, the contact feature 1830 may include four of the lobes 1942.

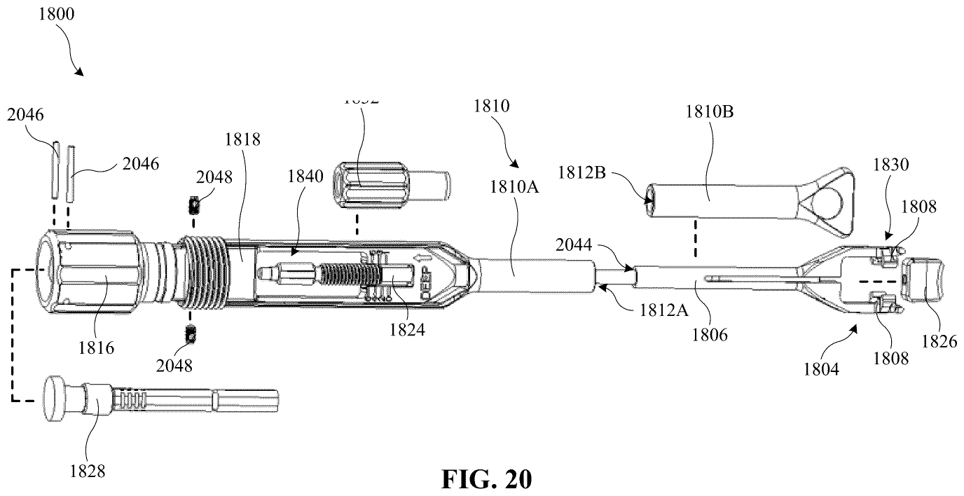

FIG. 20 is an exploded view of the cervical disc instrument 1800 to further illustrate coupling between the various components of the cervical disc instrument 1800. As described above, the jaw component 1804 may extend through a cavity 1812 defined by the sheath 1810. As shown in FIG. 20, the cavity 1812 may be defined by both the first portion 1810A and the second portion 1810B of the sheath 1810. For example, the first portion 1810A of the sheath 1810 may define a first portion 1812A of the cavity 1812 and the second portion 1810B of the sheath 1810 may define a second portion 1812B of the cavity 1812. However, in certain embodiments only the first portion 1810A or the second portion 1810B may define the cavity 1812.

The tamp 1826 may be located between the jaws 1808 and coupled to the drive shaft 1824. For example, the drive shaft 1824 may extend through both the cavity 1812 of the sheath 1810 and a cavity 2044 defined by the jaw shaft 1806 so that the drive shaft 1824 may be coupled to the tamp 1826 and also be in communication with the impact cap 1828 the extends through the sheath knob 1816.

To maintain engagement of the sheath knob 1816 during operation of the sheath 1810, the sheath knob 1816 may be coupled to the body 1818 by one or more sheath knob pins 2046 extending through the sheath knob 1816 and into the body 1818 of the cervical disc instrument 1800. Similarly, one or more retention features 2048, such as pins or plungers, may be configured to retain the drive shaft 1824 within the body 1818 (e.g., within the window 1840 of the body 1818 along the longitudinal axis 1814 of the cervical disc instrument 1800.

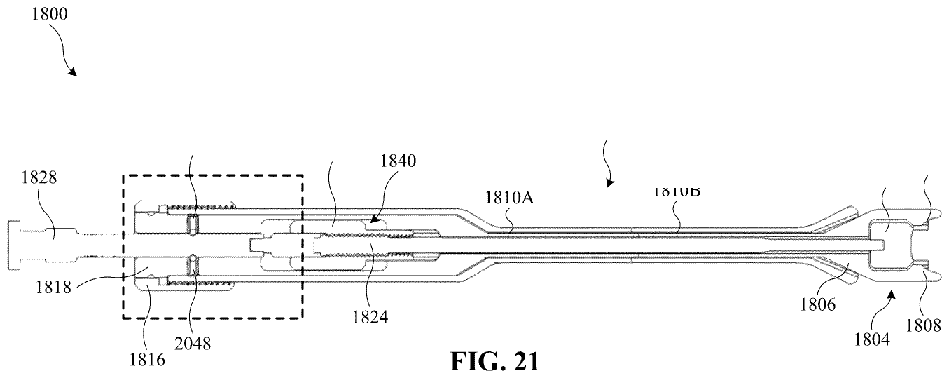

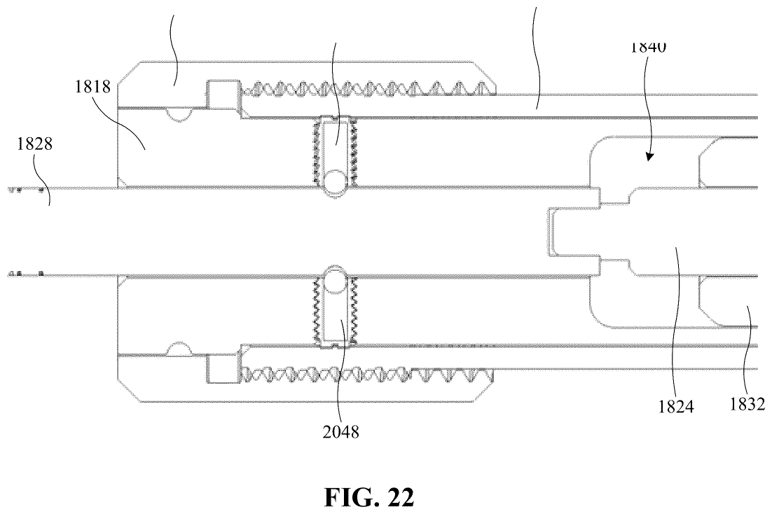

FIG. 21 is a cross-sectional view of the cervical disc instrument 1800 and FIG. 22 is a close-up view of FIG. 21 to further illustration the retention features 2048. As shown in FIGS. 21 and 22, the retention features 2048 may extend through all or a portion of the body 1818 to contact the impact cap 1828. The impact cap 1828 may have a notch or groove that may receive a respective one of the retention features 2048 to maintain a position of the impact cap 1828. Additionally, the impact cap 1828 may be coupled (e.g., threadably coupled) to the drive shaft 1824. As such, retention of the impact cap 1828 may also retain a position of the drive shaft 1824. It should be noted that a force of impact on the impact cap 1828 may be done to overcome a force exerted on the impact cap 1828 by the retention features 2048 to move the impact cap 1828 and impact the disc implant 1802.

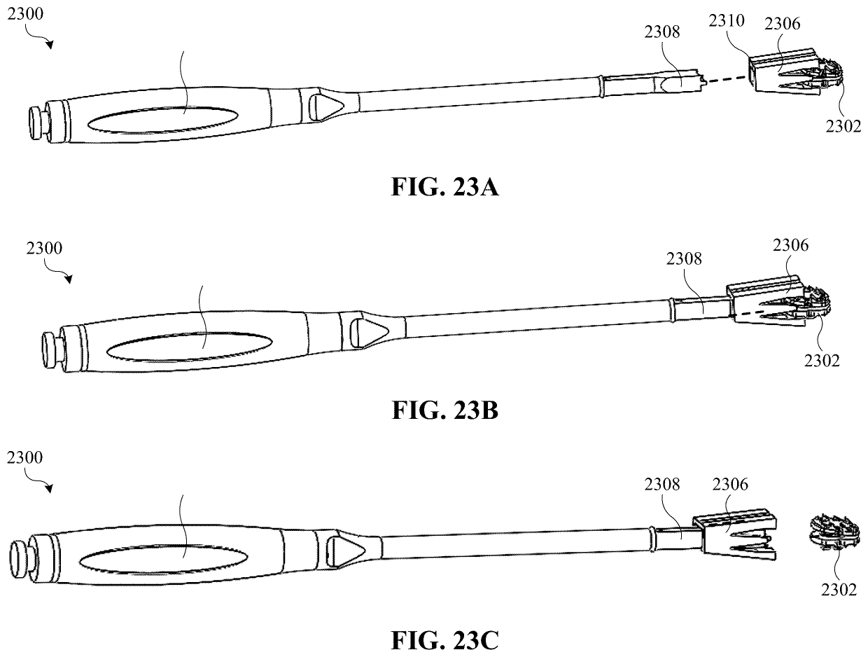

FIGS. 23A-23C illustrate perspective views of a sixth example of a cervical disc instrument 2300. The cervical disc instrument 2300 may be similar to the cervical disc instrument 1800. For example, the cervical disc instrument 2300 may also be configured to insertion of a disc implant 2302 into a disc space for impaction. As shown in FIGS. 23A-23C, the cervical disc instrument 2300 may include a handle 2304 and a clip 2306, whereby the clip 2306 may be removably coupled to a disc implant 2302.

To further describe operation of the cervical disc instrument 2300, FIG. 23A illustrates the disc implant 2302 coupled to the clip 2306 prior to the clip 2306 being coupled to the handle 2304. FIG. 23B illustrates both the disc implant 2302 and the clip 2306 coupled to the handle 2304. Additionally, FIG. 23C illustrates the clip 2306 coupled to the handle 2304 after uncoupling (e.g., release) of the disc implant 2302 from the clip 2306, such as after impact of the disc implant 2302 in the disc space.

The handle 2304 may be coupled to the clip 2306 via any engaging means or mechanism. For example, the handle 2304 may include an engaging portion 2308 that is received by a receiving portion 2310 of the clip 2306. The engaging portion 2308 may be a male portion and the receiving portion 2310 may be a female portion, or vice versa. The receiving portion 2310 may be configured to retain the engaging portion 2308 to maintain connection between the handle 2304 and the clip 2306. As such, when the disc implant 2302 is inserted into the disc space, the clip 2306 may contact a surface (e.g., a bone surface), at which point the handle 2304 may be disconnected from the clip 2306.

After disconnection of the handle 2304 the user (e.g., the surgeon) may continue tamping the disc implant 2302 (e.g., using a second tool) into the disc space until the disc implant 2302 reaches its final, intended position. Once the disc implant 2302 reaches its final position, the handle 2304 may be used to retrieve the clip 2306 and remove the clip 2306 from the disc space. It should also be noted that the disc implant 2302 may be tamped (e.g., impacted) entirely or partially with the handle 2304 connected to the clip 2306 or disconnect from the clip 2306.

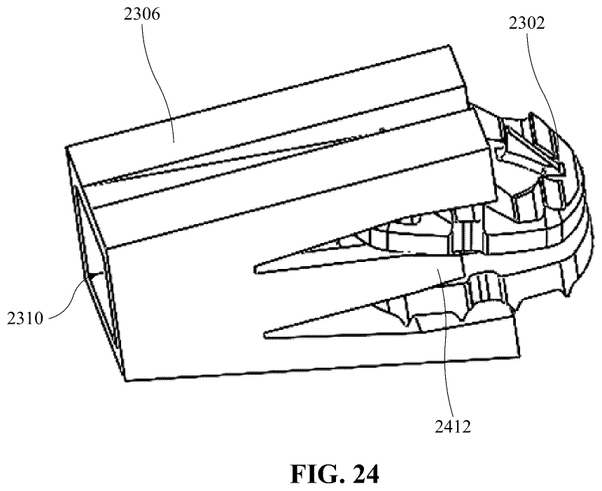

FIG. 24 is a close-up perspective view of the clip 2306. As discussed above, the clip 2306 may be removably coupled to the handle 2304 via the receiving portion 2310 engaging the engaging portion 2308 of the handle 2304. The receiving portion 2310 may be a cavity or other female feature that may receive the engaging portion 2308. Additionally, the clip 2306 may be removably coupled to the disc implant 2302 before or after the clip 2306 is connected to the handle 2304. For example, the clip 2306 may include one or more teeth 2412 or other retention features that may engage and couple to the disc implant 2302. Such retention features are not particularly limited to any size and/or shape.

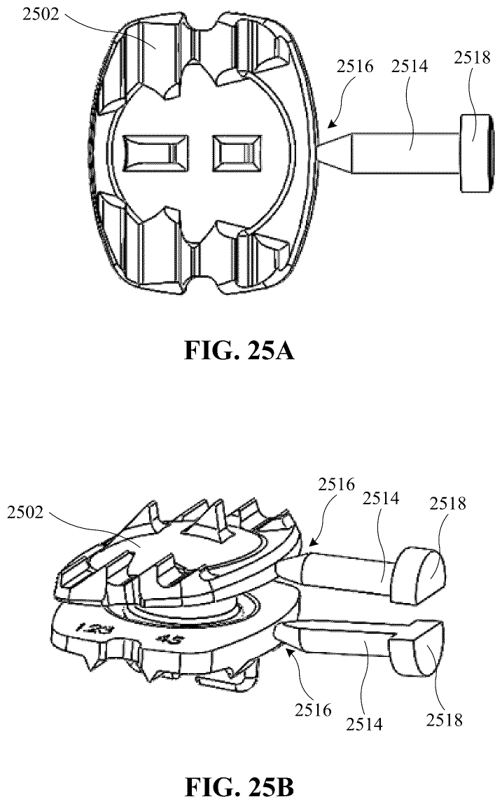

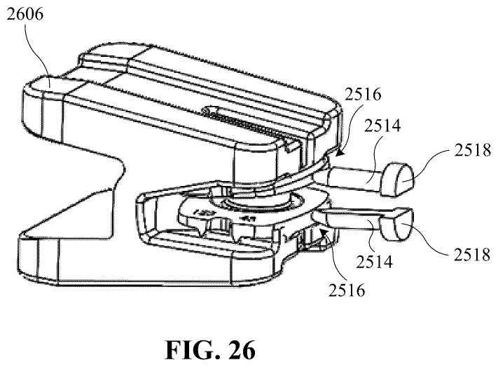

FIG. 25A illustrates a top-down view of a disc implant 2502 and FIG. 25B illustrates a perspective view of the disc implant 2502 shown in FIG. 25A. The disc implant 2502 may be removably coupled to a clip, such as the clip 2606 shown in the perspective view of FIG. 26. The clip 2606 may be similar to the clip 2306 described above and the disc implant 2502 may be similar to the disc implant 2302 described above.

To facilitate coupling of the disc implant 2502 to the clip 2606, the disc implant 2502 may include one or more posts 2514 removably coupled to the disc implant 2502. For example, the posts 2514 may be removably coupled to the disc implant 2502 via a breakaway zone 2516 such that, when the disc implant 2502 is inserted into a desired location (e.g., within the clip 2606), a tab 2518 of each of the posts 2514 may be pull to disconnect the posts 2514 from the disc implant 2502. Thus, the posts 2514 and the tabs 2518 may provide a grasping location for a user (e.g., a surgeon) to grasp the disc implant 2502, either by hand or via a tool, for proper placement of the disc implant 2502.

Although the invention has been described in detail with reference to specific embodiments, it will be apparent to one skilled in the art that various changes and modifications can be made without departing from the spirt and scope of the invention. Thus, it is intended that the invention covers the modifications and variations of this invention provided they come within the scope of the appended claims and their equivalents. It is expressly intended, for example, that all ranges broadly recited in this document include within their scope all narrower ranges which fall within the broader rangers. It is also intended that the components of the various devices disclosed above may be combined or modified in any suitable configuration.

What is claimed is:

1. A cervical disc instrument configured to insert a disc implant into a disc space, comprising:
a jaw component that includes:
a jaw shaft defining a first cavity therein; and
jaws extending from the jaw shaft and configured to grasp the disc implant;
a sheath defining a second cavity therein, wherein the jaw shaft is positioned in the second cavity;
a drive shaft configured to extend through the first cavity and the second cavity; and
a tamp coupled to the drive shaft and positioned between the jaws.

2. The cervical disc instrument of claim 1, wherein the sheath is configured to translate along a longitudinal axis of the cervical disc instrument to move from a first position, in which the sheath is spaced apart from the jaws, to a second position, in which the sheath contacts the jaws to move the jaws towards one another to grasp the disc implant.

3. The cervical disc instrument of claim 2, wherein the sheath contacts an outer surface of the jaws to move the jaw towards one another to grasp the disc implant.

4. The cervical disc instrument of claim 2, wherein a knob is coupled to the sheath, and rotation of the knob about the longitudinal axis translates the sheath along the longitudinal axis.

5. The cervical disc instrument of claim 1, further comprising:
an impact cap in communication with the drive shaft, wherein impact of the impact cap translates the tamp along a longitudinal axis of the cervical disc instrument.

6. The cervical disc instrument of claim 5, wherein the tamp is configured to translate along the longitudinal axis between the jaws to expand the jaws and extend beyond the jaws into the disc space.

7. The cervical disc instrument of claim 6, wherein the tamp is configured to expand the jaws when the jaws are grasping the disc implant to release the disc implant in a position within the disc space.

8. The cervical disc instrument of claim 1, wherein the jaw component further includes a contact feature configured to contact a surface of bone located adjacent to the disc space to prevent over-insertion of the disc implant into the disc space.

9. The cervical disc of claim 8, wherein the contact feature includes four lobes positioned adjacent to the jaws.

10. A cervical disc instrument, comprising:
a jaw component configured to releasably engage a disc implant for insertion of the disc implant into a disc space;
a sheath configured to at least partially surround the jaw component;
a drive shaft configured to extend through the sheath;
a tamp coupled to the drive shaft and configured to translate along a longitudinal axis of the cervical disc instrument to release the disc implant from the jaw component; and
a depth stop configured to prevent over-insertion of the disc implant into the disc space.

11. The cervical disc instrument of claim 10, wherein the tamp is configured to translate along the longitudinal axis to release the disc implant from the jaw component to insert the disc implant into the disc space.

12. The cervical disc instrument of claim 11, wherein the depth stop includes a knob that is threadably engaged to the drive shaft, and the depth stop is configured to contact the sheath to prevent over-extension of the tamp into the disc space.
